(19) United States
(12) Patent Application Publication
(10) Pub. No.: US 2014/0357505 A1
(43) Pub. Date: Dec. 4, 2014

(54) System And Method Of Cytomic Vascular Health Profiling

(71) Applicants: Emile R. Mohler, III, Wayne, PA (US); Jonni S. Moore, Philadelphia, PA (US); Lifeng Zhang, Voorhees, NJ (US); Wade Rogers, West Chester, PA (US); Andrew D. Bantly, Philadelphia, PA (US)

(72) Inventors: Emile R. Mohler, III, Wayne, PA (US); Jonni S. Moore, Philadelphia, PA (US); Lifeng Zhang, Voorhees, NJ (US); Wade Rogers, West Chester, PA (US); Andrew D. Bantly, Philadelphia, PA (US)

(73) Assignee: The Trustees of the University of Pennsylvania, Philadelphia, PA (US)

(21) Appl. No.: 14/123,920

(22) PCT Filed: Jun. 10, 2012
(86) PCT No.: PCT/US12/41792

§ 371 Date: Jul. 17, 2014

Related U.S. Application Data
(60) Provisional application No. 61/495,955, filed on Jun. 10, 2011.
(60) Provisional application No. 61/650,353, filed on May 22, 2012.

(51) Int. Cl. G01N 33/68

(52) U.S. Cl. CPC G01N 33/6893; G01N 2800/32
USPC 506/9; 435/29; 435/7.92; 435/7.21

(57) ABSTRACT

The present invention relates to a method of determining vascular health in a subject. The method includes the steps of obtaining a biological sample from the subject, obtaining microparticle data based on the level of at least one set of microparticles in the biological sample, obtaining progenitor cell data based on the level of at least one set of progenitor cells in the biological sample, generating a cytometric fingerprint of the biological sample based on the microparticle and progenitor cell data, and determining the vascular health of the subject based on the generated cytometric fingerprint.

### CROSS-REFERENCE TO RELATED APPLICATIONS

This application is the U.S. national phase application filed under 35 U.S.C. §371 claiming benefit to International Patent Application No. PCT/US12/41792, filed Jun. 10, 2012, which is entitled to priority under 35 U.S.C. §119(e) to U.S. Provisional Application No. 61/495,955, filed on Jun. 10, 2011, and U.S. Provisional Application No. 61/650,353, filed on May 22, 2012, the entire disclosures of which are incorporated by reference herein as if each are set forth herein in its entirety.

### STATEMENT REGARDING FEDERALLY SPONSORED RESEARCH OR DEVELOPMENT

This invention was made with government support under Grant Nos. 1RC1 HL099528-01 and 5RC1 HL099528-02 awarded by the National Institutes of Health. The government has certain rights in the invention.

### BACKGROUND OF THE INVENTION

Cardiovascular disease (CVD) is the leading cause of death in the United States; every 39 seconds an adult dies from heart attack, stroke or other cardiovascular disease (“High blood pressure and cholesterol out of control in the US.” Centers for Disease Control Web Site. http://www.cdc.gov/Features/Vitalsigns/CardiovascularDisease/. Updated Jan. 31, 2011 Accessed Feb. 6, 2012). The prevalence of CVD in the USA is already very high (36.9% of adults or about 81 million people) and is projected to increase by about 10% over the next 20 years, and by 2030 it is estimated that over 40% of adults (approximately 116 million people) will have one or more forms of CVD (Heidenreich et al., 2011, Circulation 123:933-944). Long before symptoms are clinically evident, nascent vascular disease begins as a dysfunction of endothelial cells. Symptomatic, clinical CVD events generally occur when atherosclerosis progresses to a point where obstructed blood flow causes ischemia, or when a thrombus forms from an existing plaque due to rupture or erosion (Heidenreich et al., 2011, Circulation 123:933-944).

Unfortunately, a cost-effective and accurate blood test that can determine cardiovascular health of a patient does not currently exist. Instead, cardiovascular risk is typically assessed by several circulating biomarkers, such as high-sensitivity C-reactive protein (hsCRP) and fibrinogen (Ridker, 2003, Circulation 107:363-369). As these biomarkers are generally acute-phase reactants and are thus not specific for atherosclerosis, they are recommended only for patients with intermediate risk for a cardiovascular event (Greenland et al., 2010, Circulation 122:e584-e636). Also, there are no biomarkers available in clinical practice to sensitively and accurately evaluate response to medical therapy. The Framingham-based approach to cardiovascular risk stratification does not incorporate biomarkers or genetic abnormalities and thus is limited in predictive value.

One published cardiovascular risk algorithm, the Reynolds score, includes hsCRP, but, as with Framingham, genetic background is not included in the scheme. The Evaluation of Genomic Applications in Practice and Prevention Working Group (EWG) found insufficient evidence to recommend testing for the 9p21 genetic variant or 57 other variants in 28 genes to assess risk for CVD in the general population, specifically heart disease and stroke. The EWG put forth that the magnitude of the net health benefit from the use of any of these genomic markers alone or in combination is negligible (Evaluation of Genomic Applications in Practice Prevention Working Group. 2010. Recommendations from the EGAPP Working Group: Genomic profiling to assess cardiovascular risk to improve cardiovascular health. Journal 12:839-843 810.1097/GIM.1090b1013e3181f1872c1090).

Therefore, there is an unmet clinical need for an early diagnostic test that provides a measure of cardiovascular health prior to overt CVD and an assessment of therapeutic interventions. The present invention satisfies this need via a cell-based assay assessing cardiovascular status based on the measurement of progenitor cells (PCs), such as endothelial progenitor cells (EPCs) and microparticles (MPs) in the establishment of a Vascular Health Profile (VHP).

### SUMMARY OF THE INVENTION

The present invention relates to a method of determining vascular health in a subject. The method includes the steps of obtaining a biological sample from the subject, obtaining microparticle data based on the level of at least one set of microparticles in the biological sample, obtaining progenitor cell data based on the level of at least one set of progenitor cells in the biological sample, generating a cytometric fingerprint of the biological sample based on the microparticle and progenitor cell data, and determining the vascular health of the subject based on the generated cytometric fingerprint.

In one embodiment, the level of the at least one set of microparticles is measured by flow cytometry. In another embodiment, the biological sample is a sample of whole blood. In another embodiment, the biological sample is a plasma sample. In another embodiment, the subject is a subject with psoriasis. In another embodiment, the subject is a subject with lupus. In another embodiment, the subject is a subject with known CVD risk factors. In another embodiment, the subject is a subject with no known CVD risk factors. In another embodiment, the subject is a diabetic subject. In another embodiment, the subject is a Type 1 diabetic subject. In another embodiment, the subject is a Type 2 diabetic subject. In another embodiment, the progenitor cells are endothelial progenitor cells (EPCs). In another embodiment, when the level of at least one of the microparticle sets is up-regulated, the subject is at risk of cardiovascular disease or vascular dysfunction, or of progressing cardiovascular disease or vascular dysfunction. In another embodiment, when the level of at least one of the progenitor cell sets is down-regulated, the subject is at risk of cardiovascular disease or vascular dysfunction, or of progressing cardiovascular disease or vascular dysfunction. In another embodiment, when the level of at least one of the microparticle sets is up-regulated and the level of at least one of the progenitor cell sets is down-regulated, the subject is at risk of cardiovascular disease or vascular dysfunction, or of progressing cardiovascular disease or vascular dysfunction.

The present invention also relates to a method for determining a risk associated with cardiovascular disease or vascular dysfunction in a subject. The method includes the steps of obtaining a biological sample from the subject, obtaining microparticle data based on the level of at least one set of microparticles in the biological sample, obtaining progenitor cell data based on the level of at least one set of progenitor cells in the biological sample, generating a cytometric fingerprint of the biological sample based on the microparticle and progenitor cell data, and determining the risk associated with cardiovascular disease or vascular dysfunction of the subject based on the generated cytometric fingerprint.

In one embodiment, the level of the at least one set of microparticles is measured by flow cytometry. In another embodiment, the biological sample is a plasma sample. In another embodiment, the biological sample is a sample of whole blood. In another embodiment, the subject is a subject with psoriasis. In another embodiment, the subject is a subject with lupus. In another embodiment, the subject is a subject with known CVD risk factors. In another embodiment, the subject is a subject with no known CVD risk factors. In another embodiment, the subject is a diabetic subject. In another embodiment, the subject is a Type 1 diabetic subject. In another embodiment, the subject is a Type 2 diabetic subject. In another embodiment, the progenitor cells are endothelial progenitor cells (EPCs). In another embodiment, the generated cytometric fingerprint indicates a cellular damage. In another embodiment, the generated cytometric fingerprint indicates the integrity of endothelium, a loss of endothelial repair capacity, or a combination thereof. In another embodiment, the method further comprises generating a cytometric fingerprint of a healthy control sample derived from one or more individuals, and comparing the generated cytometric fingerprint of the subject's biological sample to the cytometric fingerprint of the healthy control sample.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings. FIG. 1 is a schematic illustrating a high throughput flow cytometry assay for the determination of a vascular health profile. A blood sample is partitioned into peripheral blood mononuclear cells (PBMCs) and plasma. Progenitor cells are identified in PBMCs, while microparticles are identified in the plasma.

FIG. 2 is an illustration of FSC/SSC threshold optimization on Canto A. As depicted in FIG. 2, FSC and SSC thresholds were determined by 0.3 μm beads (Row A) or 0.3, 1 and 3 μm beads (Row B on FFC/SSC contour plot and C on SSC-W histogram) passing through the machine at medium rate with different FSC or SSC threshold. Columns D and E showed better resolutions of 3 beads on both FFC/SSC contour plot and SSC-W histogram plot with FSC threshold set to 5000 or SSC threshold to 200 (column D), and only SSC threshold set to 200 and FSC threshold off (column E). More background noise was acquired with FSC threshold set to 200 or SSC threshold to 200 (Column F). 0.3 μm beads were missing with FSC threshold set to 200 and SSC off (column G). Side scatter is a better parameter for small particles than forward scatter. FSC and SSC threshold were set to 5000 and 200 for its better resolution of 3 mixture beads on both FSC/SSC contour and SSC-W histogram plots in this study (column D).

FIG. 3 is an illustration of FSC/SSC PMT determination on Canto A. As depicted in FIG. 3, double-filtered PBS and 0.3 um beads were used to set up the FSC, SSC PMT. Less than 10 events per second was accepted when double-filtered PBS passing through the machine at medium flow rate. The threshold of FSC and SSC were set to 5000 and 200, respectively. FSC and SSC PMT were determined by the same sample running on the machine using different SSC voltage. More MPs were lost using SSC 300 and 325 and more background noise was acquired on SSC 375 and 400. SSC voltage of 350 was accepted in this study. SSC voltage of 400 data was not shown here because of too much background noise.

FIG. 4 is an illustration of window extension determination. As depicted in FIG. 4, FACS Canto A Window Extension (WE) was determined by 0.3, 1 and 3 μm beads running at medium rate form WE 0 to 7. WE 0.2 was chosen for its better resolution of 3 beads and low background noise on SSC-W histogram plots.

FIG. 5 is a comparison of window extension 0.2 and 7 in sample detection on Canto A. As depicted in FIG. 5, PFP was stained with FITC-Annexin (or CD31), Percp-Cy5.5-CD41, PE-CD105 (or CD144), APC-CD64 and run on WE 0.2 or 7. The acquisition was stopped when a fixed number of 3 μm beads (100,000) were counted. Row A was gated on less than 1 μm on dot plot with WE set to 0.2. Row B was gated on less than 1 μm on dot plot with WE set to 7. Row C was gated on less than 1 μm with WE 0.2 and D gated on less than 1 μm with WE 7 on SSC-W histogram plots. More background noise and fewer positive particles were collected using WE 7 (Row B and Row D).

FIG. 6 is an illustration of gating strategy. As depicted in FIGS. 6, 0.3, 1 and 3 μm beads were used to estimate MPs size (A and B on Canto A and C on Gallios). MPs were gated on less than 1 μm (D gated on SSC-W histogram and E on FSC/SSC dot plot on Canto and F on Gallios). For the Canto A setting, forward and side scatter thresholds were set to 5000 and 200, respectively, and the window extension was set to 0.2. For the Gallios setting, the discriminator value for FS was set to 1 and the Forward Scatter Collection Angle was W2.

FIG. 7 is an illustration of the optimization of antibodies. As depicted in FIG. 7, the same volume of antibodies used for MP detection in 500 μl of double filtered PBS was run on Canto A. Unfiltered antibodies (Row A) showed more false positive events than double-filtered antibodies (Row B). All reagents used for MPs detection should be double-filtered through 0.1-0.22 nm low protein binding filter to remove the antibody aggregates and background noise from running buffer.

FIG. 8 is a further illustration of the optimization of antibodies. As depicted in FIG. 8, 50 μl of PFP were labeled with unfiltered antibodies (Rows A and C) and double-filtered antibodies (Rows B and D). Rows A and B were gated on less than 1 μm on SSC-W histogram plot. Rows C and D were gated on less than 1 μm on FSC and SSC dot plot. Pre-filtering antibodies helps to reduce the false positive particles by removing the aggregation of antibodies.

FIG. 9 is a representation of MP detection on both Canto A and Gallios. As depicted in FIG. 9, MPs were detected on both Canto and Gallios. MPs were gated on less than 1 μm on both Canto (Row A) and Gallios (Row B). Positive MPs were determined based on fluorescence minutes one (FMO) tubes.

FIG. 10 is a comparison of BD Canto A and BC Gallios. As depicted in FIG. 10, computed Spearman rank correlations were used to compare the MP counts between the two platforms, most correlations exceeded 0.8 with the exception of CD105(+) which demonstrated a correlation of 0.6 (P<0.05). MPs counts on Gallios were two times greater than on Canto A. Row A shows the correlations of the two platforms, while Row B shows the comparison of MPs number on the two platforms.

FIG. 11 is an illustration of gating strategy for MP analysis. MPs were identified by first gating on the P1 region on the FSC/SSC plot defined by calibrator beads of less than 1 μm (A and B). The origin of the microparticles was determined by coexpression of Annexin-V and CD144 for endothelial derived MPs (C), Annexin-V and CD41 for platelet-derived MPs (D), Annexin-V and CDI4 for monocyte-derived MPs (E).

FIG. 12 is an illustration of scatter plots (with median lines) showing low density lipoprotein levels (panel A) and EPO level (panel B) compared to statin use. LDL, low density lipoprotein cholesterol; EPO, erythropoietin; H, healthy; ES, early stage diabetes; LT, long-term diabetes.

FIG. 13 is an illustration of scatter plots (with median lines) and significance of CD34+ PCs, nM of PS+ MPs by plate-based assay and ratio of nM of PS+ MPs/CD34+ PCs. P calculated by KW. H, healthy; ES, early stage diabetes; LT, long-term diabetes; MP, microparticle; PC, progenitor cell.

FIG. 14 is an illustration of median levels of ELISA plate MPs, flow cytometry measured CD34 cells and ratio. Insert table shows comparison with “non cell” atherosclertoic biomarkers.

FIG. 15 is an illustration of gating strategy for EPC analysis. A sequential gating strategy for EPCs consisted of gating (a) viable events (upper left panel), below the red dashed line, (b) cells in a size region consistent with lymphocytes (upper right panel), inside the red oval, (c) singlet events (lower left panel), inside the black polygon, and finally (d) events that are negative for the lineage markers CD3, CD19 or CD33 and dim to negative for CD45 (lower right panel), inside the lower left quadrant shown in color. The viability marker used was Propidium Iodide, detected on the PE-A channel (upper left panel). Gating was fully automatic and was applied with no operator intervention to each sample individually.

FIG. 16 is an illustration of raw (ungated) distribution of microparticles. The distribution of microparticles in an ungated, arbitrarily chosen sample is shown. All pairwise combinations of the 7 fluorescence parameters plus Side Scatter Width are shown.

FIG. 17 is also an illustration of raw (ungated) distribution of microparticles. The same sample depicted in FIG. 16 is shown after gating for particles below 1 μm.

FIG. 18 is an illustration of univariate microparticle distributions. Individual microparticle data sets were aggregated. The distribution of events with respect to each of the fluorescence parameters was plotted (x-axes) with respect to Side Scatter (y-axes). Superimposed on these distributions are kernel density estimates of the univariate distributions (black curves). Thresholds representing positive marker expression were chosen by examination of these distributions (vertical black lines).

FIG. 19 is another illustration of raw (ungated) distribution of microparticles. The same sample, depicted in FIGS. 16 and 17, is shown after gating for particles below 1 μm and for particles expressing at least one marker at a level above the threshold for positive expression (as shown in FIG. 18).

FIG. 20 is an illustration of the subset of EPCs determined by cytometric fingerprinting to be present at significantly lower concentration in DM compared with HC. The individual HC data sets are aggregated and displayed as the colored distributions in three bivariate plots using biexponential transformation. Events in the fingerprint bin that was discovered by CF as more strongly expressed in HC as compared with DM (P<0.001) are shown as black dots. The thresholds for positive expression of each of the markers shown (CD31, CD24 and CD133) were determined for each individual sample using Fluorescence Minus One (FMO) controls, and their means (solid black lines) and standard deviations (dot-dashed lines enclosing gray region) are shown.

FIG. 21 is an illustration of MP subsets present at different concentrations in DM compared with HC. CF analysis of MP distributions led to the discovery of 8 populations that are differentially expressed between HC and DM. Events in differentially expressed bins are shown as black dots superimposed on the aggregate (shown as colored distributions) of all of the individual DM data sets. Black lines represent the thresholds for positive expression determined individually for each parameter (see FIG. 18). Above each panel the phenotype of the differentially expressed subset is given. Inside each panel the cohort in which the subset is more highly expressed (either DM or HC) is shown.

FIG. 22 is an illustration of combining EPC and MP measures. In the upper panel, the vertical axis represents the ratio of MP subsets CD31^{bright}/CD41^{bright} to CD31^{dim}/CD41^{dim}. The horizontal axis represents EPC^{Rel} as described in the text. Both measures are standardized by dividing by the median among the HC group and logarithmically transformed. DM subjects are plotted as red dots, while HC subjects are plotted as blue dots. The lower two panels independently depict the two measures as box plots, in which the median is indicated by the horizontal bar, the boxes extend from the first to the third quartiles, and the whiskers extend to no more than 1.5 times the interquartile range.

FIG. 23 is an illustration of Fluorescence Minus One (FMO) analysis of the VEGF-R2 reagent. FMO control tubes were prepared by staining cells with all of the markers in the panel except one. Shown are the VEGF-R2 FMO distributions for 3 samples chosen arbitrarily. FMO thresholds were determined by first finding the boundary of the main negative cluster in the 2D kernel density estimate for the distribution of VEGF-R2 vs Side Scatter Area (red ovals), and then finding the horizontal tangent to this boundary (red dashed lines). Notice that in these samples there are significant numbers of events above the FMO threshold. Moreover, these events frequently appear to form clusters well removed from the negative population. These represent “false positive” events, because there was no VEFG-R2 reagent in these control tubes, so positive expression is due to something other than actual expression of VEFG-R2 receptors on these cells. Consequently, actual expression of VEGF-R2 cannot be reliably ascertained, especially when the target events are rare, as in the case of EPCs.

FIG. 24 is an illustration of the differential expression of microparticle phenotypes. Shown are boxplots representing differential expression between Diabetic Mellitus and Healthy Control of the microparticle subsets described in Table 6. Each box plot shows the median and first and third quartiles of the class-specific distributions.

### DETAILED DESCRIPTION

The present invention relates to a system and method of profiling vascular health. The systems and methods of the present invention include a cell-based assay for assessing cardiovascular status based on the measurement of a various PCs (such as EPCs) and MPs. This “cytomic” approach, utilizing the power of systems biology in combination with highly sensitive high dimensional flow cytometry, is a reflection of genetic and environmental influences on cardiovascular health, integrated at the cellular level and targeted to cells (and subcellular particles) that play active roles in endothelial function.

In one aspect of the present invention, the systems and methods utilize a broad and comprehensive cell surface marker panel with an unbiased analysis scheme using cytometric fingerprinting to evaluate differences between patients with diabetes mellitus (DM) and healthy controls (HC). Unlike previous studies, which only observe levels of either MPs or EPCs alone, by obtaining both MP and EPC samples, the balance of how vascular dysfunction (through the levels of MP) and reparative capacity (through the levels of EPC) interact, can be observed and provide a significantly improved diagnosis and/or determination of risk.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of subjects, organisms, tissues, cells or components thereof, refers to those subjects, organisms, tissues, cells or components thereof that differ in at least one observable or detectable characteristic (e.g., age, treatment, time of day, etc.) from those subjects, organisms, tissues, cells or components thereof that display the “normal” (expected) respective characteristic. Characteristics which are normal or expected for one cell or tissue type, might be abnormal for a different cell or tissue type.

The term “assessing” includes any form of measurement, and includes determining if an element is present or not. The terms “determining,” “measuring,” “evaluating,” “assessing,” and “assaying” are used interchangeably and include quantitative and qualitative determinations. Assessing may be relative or absolute. “Assessing the presence of” includes determining the amount of something present, and/or determining whether it is present or absent.

As used herein, the term “biomarker” is a biological entity such as a cell or group of cells or a fragment or fragments thereof, a protein or a fragment thereof, including a polypeptide or peptide that may be isolated from, or measured in or on the biological sample, which is differentially present in a sample taken from a subject having established or potentially clinically significant CVD as compared to a comparable sample taken from an apparently normal subject that does not have CVD. A biomarker can be an intact cell or molecule, or it can be a portion thereof that may be partially functional or recognized, for example, by a specific binding protein or other detection method. A biomarker is considered to be informative if a measurable aspect of the biomarker is associated with the presence or risk of CVD in a subject in comparison to a predetermined value or a reference profile from a control population. Such a measurable aspect may include, for example, the presence, absence, amount, or concentration of the biomarker, or a portion thereof, in the biological sample, and/or its presence as a part of a profile of more than one biomarker. A measurable aspect of a biomarker is also referred to as a feature. A feature may be a ratio or other such mathematically defined relationship of two or more measurable aspects of biomarkers. A biomarker profile comprises at least one measurable feature, and may comprise two, three, four, five, 10, 20, 30 or any number of features. The biomarker profile may also comprise at least one measurable aspect of at least one feature relative to at least one external or internal standard.

As used herein, the term “cytometric fingerprint” refers to a representation of the multivariate probability distribution of a plurality of cells, microparticles or other objects as measured in a flow cytometer. In flow cytometry instrumentation, each cell or microparticle is typically characterized by not less than two measured variables, and often by as many as twenty or more measured variables. The flow cytomtetric measurement of a plurality of cells can thus be characterized by a distribution in a hyperspace defined by as many dimensions as the number of measurement variables. A cytometric fingerprint is a compact representation of this multivariate probability distribution in the form of a vector of numbers, each number representing the density of the distribution function in a particular sub-region of the multivariate space.

As used herein, the term “cardiovascular disease” or “CVD,” generally refers to heart and blood vessel diseases, including atherosclerosis, coronary heart disease, cerebrovascular disease, and peripheral vascular disease. Cardiovascular disorders are acute manifestations of CVD and include myocardial infarction, stroke, angina pectoris, transient ischemic attacks, and congestive heart failure. Cardiovascular disease, including atherosclerosis, usually results from the build-up of fatty material, inflammatory cells, extracellular matrix and plaque.

As used herein, the term “data” in relation to one or more biomarkers, or the term “biomarker data” generally refers to data reflective of the absolute and/or relative abundance (level) of a product of a biomarker in a sample. As used herein, the term “dataset” in relation to one or more biomarkers refers to a set of data representing levels of each of one or more biomarker products of a panel of biomarkers in a reference population of subjects. A dataset can be used to generate a formula/classifier of the invention. According to one embodiment the dataset need not comprise data for each biomarker product of the panel for each individual of the reference population. For example, the “dataset” when used in the context of a dataset to be applied to a formula can refer to data representing levels of products of each biomarker for each individual in one or more reference populations, but as would be understood can also refer to data representing levels of products of each biomarker for 99%, 95%, 90%, 85%, 80%, 75%, 70% or less of the individuals in each of said one or more reference populations and can still be useful for purposes of applying to a formula.

Diabetes mellitus (DM) is a severe, chronic form of diabetes caused by insufficient production of insulin and resulting in abnormal metabolism of carbohydrates, fats, and proteins. The disease is characterized by increased sugar levels in the blood and urine, excessive thirst, frequent urination, acidosis, and wasting. The condition is exacerbated by obesity and an inactive lifestyle. This disease often has no symptoms, is usually diagnosed by tests that indicate glucose intolerance, and is treated with changes in diet and an exercise regimen. Diabetes mellitus is associated with high risk of cardiovascular complications including diseases of coronary, peripheral, and carotid arteries. As demonstrated herein, DM was used as a model system of vascular disease and results of the vascular health profile of the present invention from a diabetic cohort was compared to age and gender-similar healthy controls (HC) to discover biologically informative markers to aid in detection and treatment of vascular complications.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A “formula,” “algorithm,” or “model” is any mathematical equation, algorithmic, analytical or programmed process, or statistical technique that takes one or more continuous or categorical inputs (or “parameters”) and calculates an output value, sometimes referred to as an “index” or “index value.” Non-limiting examples of “formulas” include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations. Of particular use in combining CVD markers and other biomarkers are linear and non-linear equations and statistical classification analyses to determine the relationship between levels of CVD markers detected in a subject sample. In panel and combination construction, of particular interest are structural statistical classification algorithms, and methods of risk index construction, utilizing pattern recognition features, including established techniques such as cross-correlation, Principal Components Analysis (PCA), factor rotation, Logistic Regression (LogReg), Linear Discriminant Analysis (LDA), Support Vector Machines (SVM), Random Forest (RF), Partial Least Squares, Sparse Partial Least Squares, Flexible Discriminant Analysis, Recursive Partitioning Tree (RPART), as well as other related decision tree classification techniques, Nearest Shrunken Centroids (SC), stepwise model selection procedures, Kth-Nearest Neighbor, Boosting or Boosted Tree, Decision Trees, Neural Networks, Bayesian Networks, Support Vector Machines, and Hidden Markov Models, and others. Other techniques may be used in survival and time to event hazard analysis, including Cox, Weibull, Kaplan-Meier and Greenwood models well known to those of skill in the art.

“Increased risk of developing CVD” is used herein to refer to an increase in the likelihood or possibility of a subject developing CVD. This risk can be assessed relative to an individual's own risk, or with respect to a reference population that does not have clinical evidence of CVD. The reference population may be representative of the individual with regard to approximate age, age group and/or gender.

“Increased risk of progressing CVD” is used herein to refer to an increase in the likelihood or possibility of a subject having CVD to have progressing CVD. This risk can be assessed relative to an individual's own risk, or with respect to a reference population that does not have clinical evidence of CVD. The reference population may be representative of the individual with regard to approximate age, age group and/or gender.

As used herein, an “instructional material” includes a publication, a recording, a diagram, or any other medium of expression which can be used to communicate the usefulness of a compound, composition, vector, biomarker or delivery system of the invention in the kit for effecting determining or assessing risk of the various diseases or disorders recited herein. The instructional material of the kit of the invention can, for example, be affixed to a container which contains the identified compound, composition, vector, biomarker or delivery system of the invention or be shipped together with a container which contains the identified compound, composition, vector, biomarker or delivery system. Alternatively, the instructional material can be shipped separately from the container with the intention that the instructional material and the compound, composition, vector, biomarker or delivery system be used cooperatively by the recipient.

The “level” of one or more biomarkers means the absolute or relative amount or concentration of the biomarker in the sample.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

“Microparticles” (MPs) as used herein are 0.1-1 μm plasma particles shed from eukaryotic cells that are formed by exocytic budding due to activation or apoptosis, and are indicative of cell damage. As contemplated herein, MPs may be endothelial MPs (EMPs), platelet MPs (PMPs) and/or monocyte MPs (MMPs). MPs contain miRNA, proteins and other antigens from their parent cell and are often pro-coagulative and pro-inflammatory.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “predetermined value” refers to the amount of one or more biomarkers in biological samples obtained from the general population or from a select population of subjects. For example, the select population may be comprised of apparently healthy subjects, such as individuals who have not previously had any sign or symptoms indicating the presence of CVD. In another example, the predetermined value may be comprised of subjects having established CVD. In another example, the predetermined value may be comprised of subjects having DM. The predetermined value can be a cut-off value, or a range. The predetermined value can be established based upon comparative measurements between apparently healthy subjects and subjects with established CVD, ES or LT or DM, as described herein.

“Progenitor cell” (PC) as used herein may include any type of PC understood by those skilled in the art, including proangiogenic cells (PACs), endothelial progenitor cells (EPCs) and circulating hematopoietic stem and progenitor cells (CHSPCs). As demonstrated herein, a population of cells was discovered that is phenotypically consistent with common definitions of EPCs, but also with CHSPCs. For purposes of brevity and clarity, such cells are referred to herein simply as PCs.

“Sample” or “biological sample” as used herein means a biological material isolated from an individual. The biological sample may contain any biological material suitable for detecting the desired biomarkers, and may comprise cellular and/or non-cellular material obtained from the individual.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

### DESCRIPTION

Cell-based systems analyses, also known as ‘cytomics’, integrates the biologic consequences of environmental and genetic cardiovascular risk factors. There is an unmet clinical need to develop such assays that could be used routinely to guide medical therapy and risk assessment. The systems and methods of the present invention provide a comprehensive insight into vascular health by using pattern discovery computational methods to analyze characteristics of several targets, including populations of vascular microparticles (recently identified as robust biomarkers of vascular health) and endothelial progenitor cells. For example, asymptomatic patients can be evaluated for cardiovascular risk, and symptomatic patients can be monitored longitudinally. These capabilities realize the main goals of personalized medicine.

As contemplated herein, the systems and methods of the present invention include an early diagnostic test that provides a measure of cardiovascular health prior to overt CVD, and an assessment of therapeutic interventions. It should be appreciated that the present invention may be used for the measure of any parameter of cardiovascular health, and may be suitable for the detection and determination of risk for any CVD as would be understood by those skilled in the art.

For example, diabetes mellitus (DM) is associated with high risk of cardiovascular complications including diseases of coronary, peripheral, and carotid arteries. Patients with type 2 DM have a 2- to 4-fold increase in the risk of CAD and PAD (Beckman et al., 2002, JAMA 287:2570-2581). As a result, it is believed that blood samples from patients with long-term type 2 DM and with clinically apparent atherosclerosis will have an abnormal vascular health profile different from non-diabetic control subjects. As demonstrated herein, DM was used as a model system of vascular disease and results of the vascular health profile of the present invention from a diabetic cohort was compared to age and gender-similar healthy controls (HC) to discover biologically informative markers to aid in detection and treatment of vascular complications.

### Progenitor Cells and Methods of Measurement

As contemplated herein, the present invention includes the identification and gating of various progenitor cells (PCs) in a sample. PCs are defined herein to include PACs, EPCs and/or CHSPCs and any other progenitor cell type associated with CVD. These cells are mediators of reparative capacity, and while a precise phenotypic definition of such cells has yet to be determined, they are thought to participate in angiogenesis either through structural development or paracrine action to support vascular growth.

EPCs are bone marrow-derived cells that mobilize into circulation in response to endogenous (e.g., from ischemic tissue, tumor cells) or exogenous (e.g., statins) signals. In brief, the physiological function of EPCs contributes to vascular homeostasis, which is crucial to prevent the pathogenesis of various diseases with vascular injury (Möbius-Winkler et al., 2009, Cytometry Part A 75A:25-37).

Surface markers often used to identify EPCs with flow cytometry include CD133, CD34 and VEGF-R2 (also called KDR) (Möbius-Winkler et al., 2009, Cytometry Part A 75A:25-37; Hirschi et al., 2008, Arterioscler Thromb Vasc Biol. 28:1584-1595; Khan et al., 2005, Cytometry B Clin Cytom 64:1-8). A recent study by Estes et. al. defined a population of cells with in vitro hematopoietic colony forming activity and multilineage engraftment in NOD/SCID mice with the phenotype CD31⁺, CD34⁺, CD133⁺ and CD45^{dim-negative} (Estes et al., 2010, Cytometry Part A 77A:831-839), to which they refer as circulating hematopoietic stem and progenitor cells (CHSPCs). As contemplated herein, there is no limitation to the number and type of surface markers used to characterize a PC or population of PCs, as would be understood by those skilled in the art.

PCs can be measured by any suitable method understood by those skilled in the art. For example, the cells may be measured by a high throughput method. In one embodiment, the cells are measured by flow cytometry. In another embodiment, the cells are measured by an ELISA based technique. In yet another embodiment, the cells are measured by a plate based capture assay. In yet another embodiment, the cells measured by flow cytometry are represented using cytometric fingerprinting. In some embodiments, the cells are measured by a plurality of methods in any combination. For example, flow cytometry, an ELISA based technique, and a plate based capture assay or any combination thereof may be used.

### Microparticles and Methods of Measurement

Microparticles (MPs) are 0.1-1 μm plasma particles shed from eukaryotic cells that are formed by exocytic budding due to activation or apoptosis, and are indicative of cell damage. As contemplated herein, MPs may be endothelial MPs (EMPs), platelet MPs (PMPs) and/or monocyte MPs (MMPs).

MPs contain miRNA, proteins and other antigens from their parent cell and are often pro-coagulative and pro-inflammatory. The role of MPs in coagulation and inflammation is an important part of atherosclerotic pathophysiology, making MPs especially attractive as potential biomarkers of vascular health. Indeed, many studies have demonstrated elevated cell-specific MPs in conditions of vascular dysfunction (Tushuizen et al., 2011, Arterioscler Thromb Vasc Biol 31:4-9). Additionally, MPs can prevent apoptosis in their parent cell by ‘exporting’ pro-apoptotic compounds such as Caspase 3, thereby lowering cytosolic levels (Hussein et al., 2007, Thromb Haemost 98(5):1096-107). Still, MPs are significantly elevated in patients with acute coronary syndromes compared to patients with stable anginal symptoms (Bernal-Mizrachi et al., 2003, American Heart Journal 145:962-970), and are a robust predictor of secondary myocardial infarction or death (Sinning et al., 2011, European Heart Journal 32:2034-2041). They are also elevated following acute ischemic stroke/cerebrovascular accident (Jung et al. 2009. Ann Neurol. 66(2):191-9). In a prospective observational study of diabetic patients, elevated MPs robustly predicted the presence of coronary lesions, and proved to be a more significant independent risk factor than length of diabetic disease, lipid concentrations, or the presence of hypertension (Koga et al., 2005, J Am Coll Cardiol 45:1622-1630). Circulating leukocyte-derived MPs were predictive of subclinical atherosclerosis and plaque numbers in 216 asymptomatic subjects (Chironi et al., 2006, Arterioscler Thromb Vasc Biol. 26:2775-2780). As contemplated herein, MP presence, number, and type may be used as biomarkers and/or as a component of the sensitive and specific vascular health profile assay of the present invention, with clinical utility in predicting atherosclerotic risk in asymptomatic patients.

These submicron particles are released into circulation, carrying with them an array of surface markers, used to identify their cellular source. Exposed membrane phosphatidylserine (PS) and tissue factor, along with a plethora of other surface molecules and cytoplasmic components, including nuclear material, enable MPs to impact on a variety of biological functions, including coagulation, thrombosis, and angiogenesis.

MPs can be measured by any suitable method understood by those skilled in the art. For example, MPs may be measured by a high throughput method. In one embodiment, the cells are measured by flow cytometry, as demonstrated in the various Examples. As contemplated herein, there is no limitation to the number and type of surface markers used to characterize a MP or population of MPs, as would be understood by those skilled in the art.

### Cytomeric Fingerprinting

Cytometric Fingerprinting (Rogers et al., 2008, Cytometry Part A 73A:430-441; Rogers & Holyst, 2009, Adv Bioinformatics:193947) (CF) provides a means to rapidly analyze high-dimensional, high-content flow cytometry data without investigator or system bias. CF breaks a multivariate distribution into a large number of non-overlapping regions, referred to herein as “bins,” that fully span the space, resulting every event recorded in the dataset is found in one of the bins. Given the set of bins, CF assigns each event to a bin, counts the number of events in each bin, and represents the full multivariate distribution for a sample as a flattened vector, referred to herein as a “fingerprint,” of the number of events per bin. As demonstrated herein, the fingerprint may be regarded as a complete micro-gating of the data, with each gate, or bin, being tagged.

As also demonstrated herein, the multivariate probability distribution functions for multiple samples can be compared using straightforward statistical analysis methods. For example, one can search for bins that are significantly up-regulated and/or down-regulated in a group of samples as compared with another group of samples using methods similar to those now routinely employed for the analysis of gene expression data (Boscolo et al., 2008, IEEE/ACM Transactions on 5(1):15-24). Therefore, the use of CF not only enables a “datamining” approach to the analysis of flow cytometric data, whereby disease-related or treatment-related alterations of the multivariate distributions are discovered directly from the data, but also builds a bridge to integrative analysis with other technologies (e.g., Doring et. al. (Döring et al., 2012, Arterioscler Thromb Vasc Biol. 32:182-195)). This stands in contrast to conventional analysis methods that rely on investigator-defined gates, which represent hypotheses of specific regions of multivariate space that might vary due to disease or treatment. Thus, CF not only eliminates the possibility of investigator bias (which may be unintentionally introduced by gating subjectivity), but also allows for a comprehensive analysis of all multivariate regions, not just those that are pre-defined by investigators' expectations.

### Methods of the Invention

As described herein, the present invention utilizes an array of cell-based biomarkers in the determination of vascular health of a subject. The method can be generally described as shown in FIG. 1, wherein a biological sample, such as blood, is collected from a subject. As contemplated herein, the biological sample of the subject and used in performance of the methods described herein may be blood, sera, plasma, or any other suitable fluid, tissue or cellular sample as would be understood by those skilled in the art. Next, at least one PC and at least one MP is measured by a high throughput method, such as flow cytometry. Next, a cytometric fingerprinting of the flow cytometry data is performed to categorize the data into a plurality of categories without investigator or system bias, such that vascular health profile of the identified biomarkers is obtained. Using this profile, significantly up-regulated and/or down-regulated biomarkers in a group of samples as compared with another group of samples can be identified and used in the determination or prediction of CVD or risk of increasing or progressing CVD in the subject.

In another embodiment, the present invention relates to a method of determining vascular health in a subject. The method includes the steps of obtaining a biological sample from the subject, obtaining microparticle data based on the level of at least one set of microparticles in the biological sample, obtaining progenitor cell data based on the level of at least one set of progenitor cells in the biological sample, generating a cytometric fingerprint of the biological sample based on the microparticle and progenitor cell data, and determining the vascular health of the subject based on the generated cytometric fingerprint.

In another embodiment, a comprehensive panel was employed, in which cells were first selected based on size. Then, cells belonging to the mature hematopoietic lineage were removed by gating out CD3⁺, CD19⁺, CD33⁺ and CD45^{bright} cells. Then, using cytometric fingerprinting, the remaining population was subdivided and subjected to statistical evaluation, without any predetermined bias, to discover if there were populations of cells differentially expressed between the DM patients and HC.

In one embodiment, the invention relates to methods for determining the risk of cardiovascular disease incidence or vascular dysfunction in a diabetic subject by measuring the relationship of microparticles to progenitor cells. In one embodiment, the method determines or is predictive of an increased risk of developing CVD. In another embodiment, the method determines or is predictive of an increased risk of progressing CVD. For example, the method includes the steps of obtaining a biological sample from the subject, obtaining microparticle data based on the level of at least one set of microparticles in the biological sample, obtaining progenitor cell data based on the level of at least one set of progenitor cells in the biological sample, generating a cytometric fingerprint of the biological sample based on the microparticle and progenitor cell data, and determining the risk associated with cardiovascular disease or vascular dysfunction of the subject based on the generated cytometric fingerprint.

In another embodiment, the invention includes a method for determining the vascular health in a subject, where the method includes the steps of obtaining a biological sample from the subject, and determining the type and/or level of microparticles relative to the level of progenitor cells in the sample, wherein the relative level of microparticles to progenitor cells indicates a risk associated with cardiovascular disease or vascular dysfunction in the subject, thereby determining vascular health in said subject.

In another embodiment, the invention includes a method for determining the risk associated with cardiovascular disease or vascular dysfunction in a diabetic subject, the method including the steps of obtaining a biological sample from said subject, determining the level of microparticles relative to the level of progenitor cells in the sample, wherein the relative level of microparticles to progenitor cells indicates the risk associated with cardiovascular disease or vascular dysfunction in said subject. In one embodiment, the diabetic subject is a Type 1 diabetic subject. In another embodiment, the diabetic subject is a Type 2 diabetic subject.

In yet another embodiment, the invention includes a method for determining diabetes associated risk in a subject, the method comprising the steps of obtaining a biological sample from the subject, determining the level of microparticles relative to the level of progenitor cells in said sample, wherein the relative level of microparticles to progenitor cells indicates a risk associated with cardiovascular disease or vascular dysfunction in the subject, thereby determining diabetes associated risk in the subject.

In some embodiments, the methods of the invention include the step of determining the level of microparticles relative to the level of PACs or EPCs.

In one embodiment, the relative level of microparticles to PCs, PACs or EPCs indicates cellular damage. In another embodiment, the relative level of microparticles to PCs, PACs or EPCs indicates the integrity of endothelium, a loss of endothelial repair capacity, or a combination thereof.

In one embodiment, the comparison of levels of microparticles and progenitor cells is a ratio of microparticles to progenitor cells. In some embodiments, this ratio can be directly associated with aortic pulse wave velocity (aPWV), providing a functional link between plasma cholesterol levels, MPs, PACs, endothelial injury, and arterial stiffness.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### Detection of Circulating MPs by Flow Cytometry

As explained previously, flow cytometry (FCM) can be used for the detection of microparticles (MPs) in blood. This technology enables measurement of thousands of MPs in one sample, with the simultaneous determination of multiple markers identifying various MP subsets. The very small size of MPs (0.1 to 1.0 μm) makes their detection at the limit of size resolution of standard flow cytometers. Thus, accurate detection requires exquisite attention to detail in specimen preparation, sample acquisition, and data analysis. As demonstrated herein, the detection of MPs in human plasma in patients was optimized using the BD Biosciences FACS Canto A and Beckman Coulter Gallios.

The following materials and methods were used in Example 1:

Platelet free plasma (PFP) was obtained by two step centrifugation of heparinized blood from 37 healthy subjects (1500 g for 15 min and 13,000 g for 2 minutes). 50 μl of PFP was labeled with double filtered 5 μl FITC-CD31 (555445, BD), 5 n1 PE-CD144 (560410, BD), 2.5 μl APC-CD64 (CD6405, Invitrogen) and 5 μl PerCP-Cy5.5-CD41a (340930, BD) or 2.5 n1 of FITC-Annexin V (556570, BD), 1.25 μl PE-CD105 (560839, BD) and 5 μl Percp-Cy5.5-CD3 (340949, BD). FMO tubes were used to set up the negative gates.

After 30 minutes of incubation at room temperature, 50 μl of 3 μm beads (BCP-30-5, Spherotech) and double filtered PBS or Annexin buffer were added to the tube to make the total final volume to 500 μl. Samples were then run on FACS Canto A (BD) and Gallios (Beckman Coulter).

For data acquisition and analysis, forward and side scatter threshold, photomultiplier tube (PMT) voltage and window extension (WE) were optimized to detect 0.1-1 μm particles using 0.3, 1 and 3 μm calibration beads. MP were analyzed in a protocol with both forward scatter (FSC) and side scatter (SSC) in logarithmic mode. Standard beads 0.3 (Sigma), 1 and 3.0 nm diameter (Spherotech) were used for estimation of MP size. Events with 0.3 to 1.0 nm size on SSW or FSC-SSC graphs were gated as MPs. The acquisition was stopped when a fixed number of 3 μm beads (200,000) were counted for both Canto and Gallios. Analysis was performed with DiVa version 6.1.2 on the BD FACS Canto A and Kaluza version 1.1 on the BC Gallios.

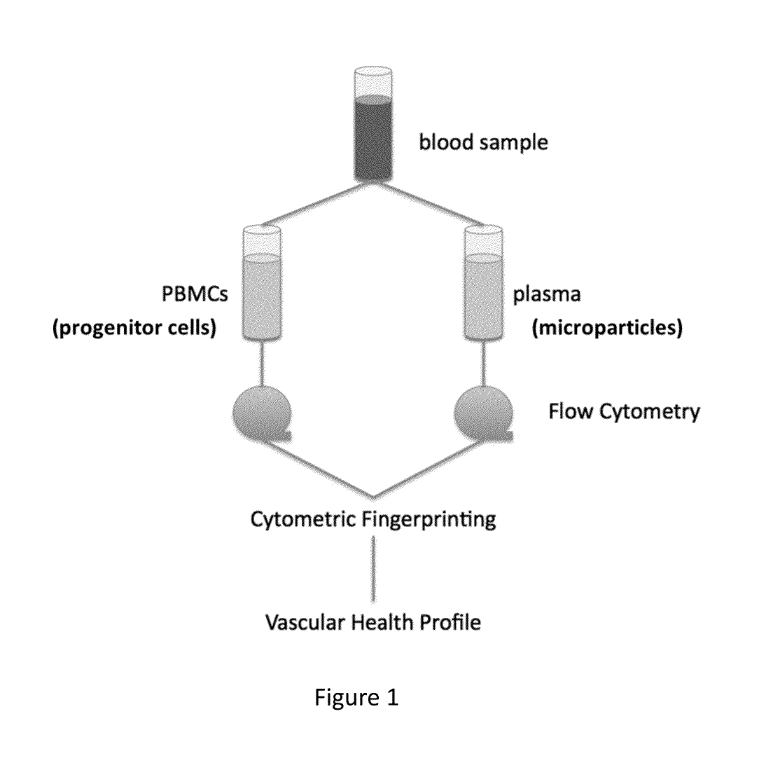

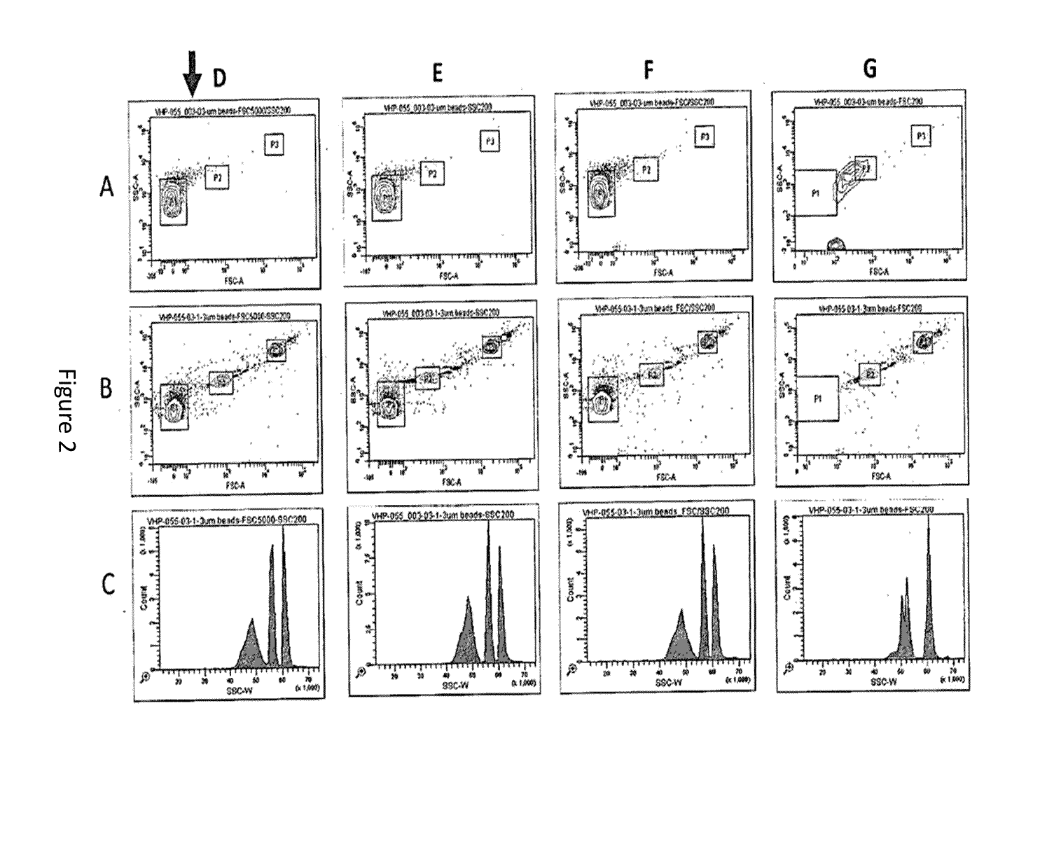

As illustrated in FIG. 2, FSC and SSC thresholds were determined by 0.3 μm beads (Row A) or 0.3, 1 and 3 μm beads (Row B on FFC/SSC contour plot and C on SSC-W histogram) passing through the machine at medium rate with different FSC or SSC threshold. Columns D and E showed better resolutions of 3 beads on both FFC/SSC contour plot and SSC-W histogram plot with FSC threshold set to 5000 or SSC threshold to 200 (column D), and only SSC threshold set to 200 and FSC threshold off (column E). More background noise was acquired with FSC threshold set to 200 or SSC threshold to 200 (Column F). 0.3 nm beads were missing with FSC threshold set to 200 and SSC off (column G). Side scatter is a better parameter for small particles than forward scatter. FSC and SSC threshold were set to 5000 and 200 for its better resolution of 3 mixture beads on both FSC/SSC contour and SSC-W histogram plots in this study (column D).

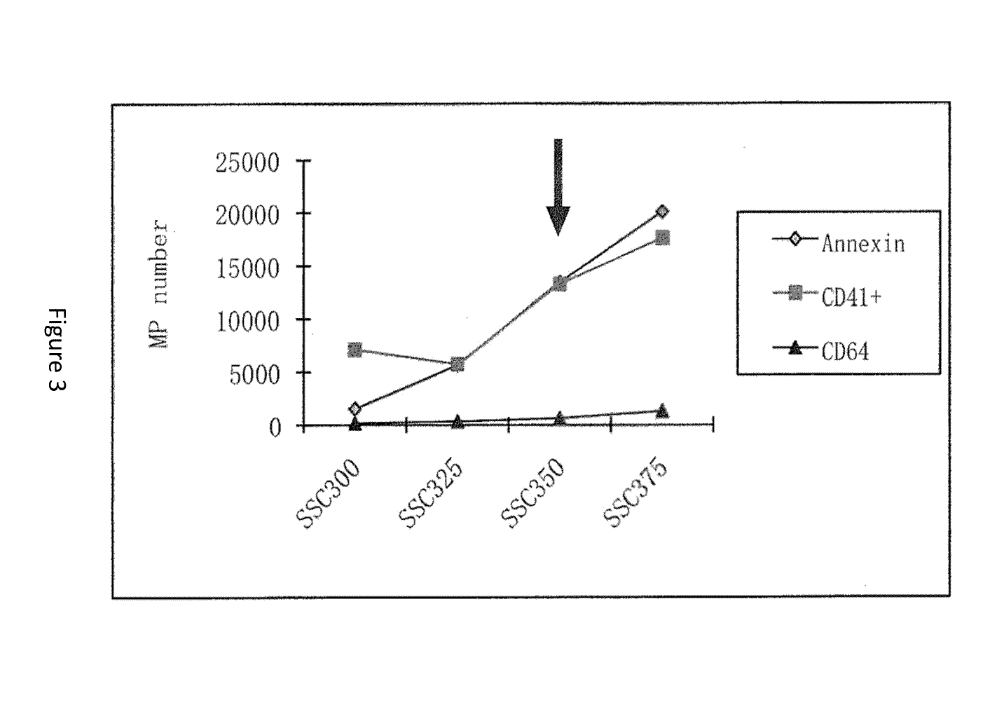

As illustrated in FIG. 3, double-filtered PBS and 0.3 um beads were used to set up the FSC, SSC PMT. Less than 10 events per second was accepted when double-filtered PBS passing through the machine at medium flow rate. The threshold of FSC and SSC were set to 5000 and 200, respectively. FSC and SSC PMT were determined by the same sample running on the machine using different SSC voltage. More MPs were lost using SSC 300 and 325 and more background noise was acquired on SSC 375 and 400. SSC voltage of 350 was accepted in this study. SSC voltage of 400 data was not shown here because of too much background noise.

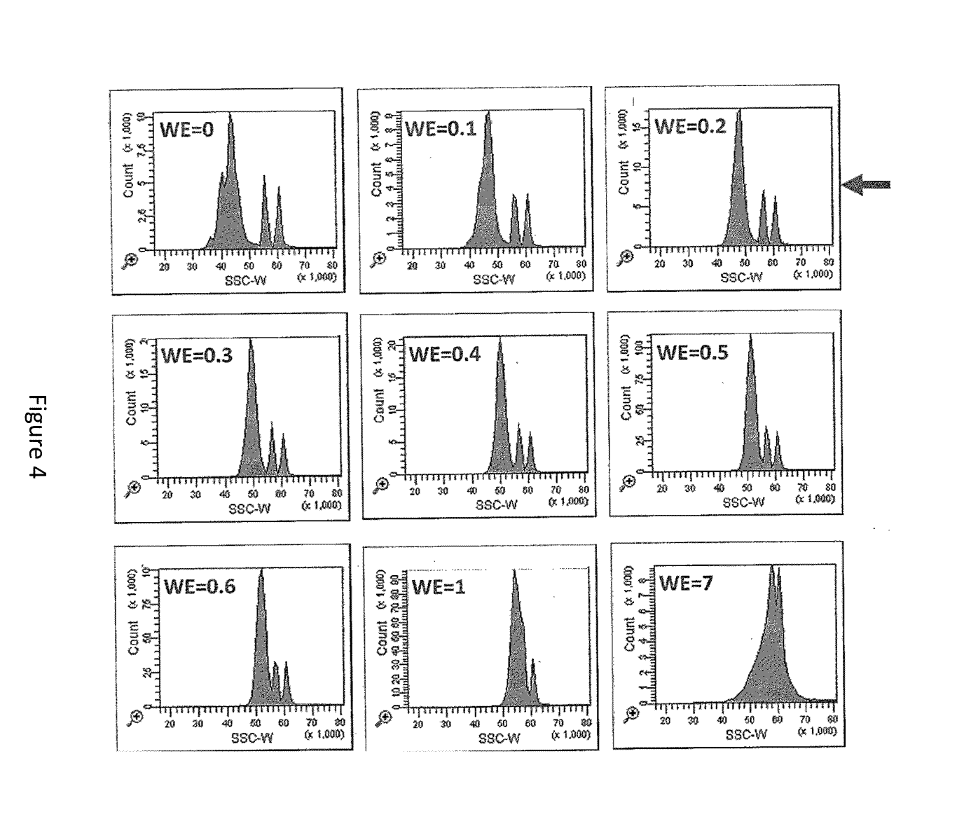

As illustrated in FIG. 4, FACS Canto A Window Extension (WE) was determined by 0.3, 1 and 3 μm beads running at medium rate form WE 0 to 7. WE 0.2 was chosen for its better resolution of 3 beads and low background noise on SSC-W histogram plots.

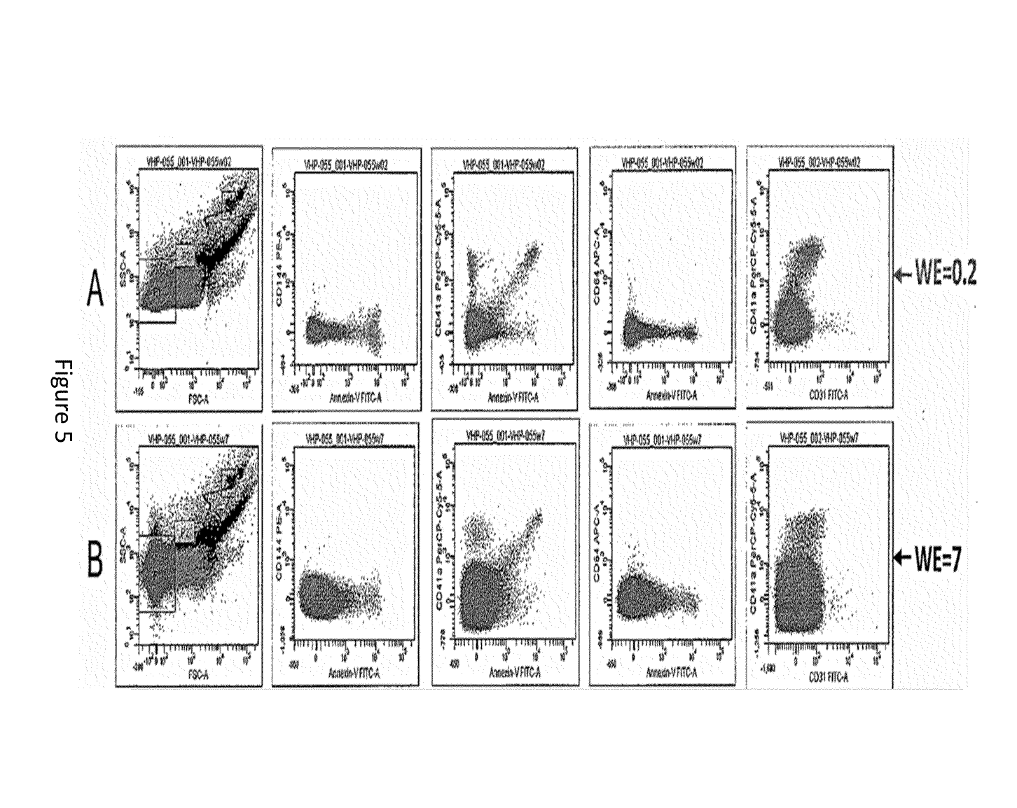

As illustrated in FIG. 5, PFP was stained with FITC-Annexin (or CD31), Percp-Cy5.5-CD41, PE-CD105 (or CD144), APC-CD64 and run on WE 0.2 or 7. The acquisition was stopped when a fixed number of 3 μm beads (100,000) were counted. Row A was gated on less than 1 μm on dot plot with WE set to 0.2. Row B was gated on less than 1 μm on dot plot with WE set to 7. Row C was gated on less than 1 μm with WE 0.2 and D gated on less than 1 μm with WE 7 on SSC-W histogram plots. More background noise and fewer positive particles were collected using WE 7 (Row B and Row D).

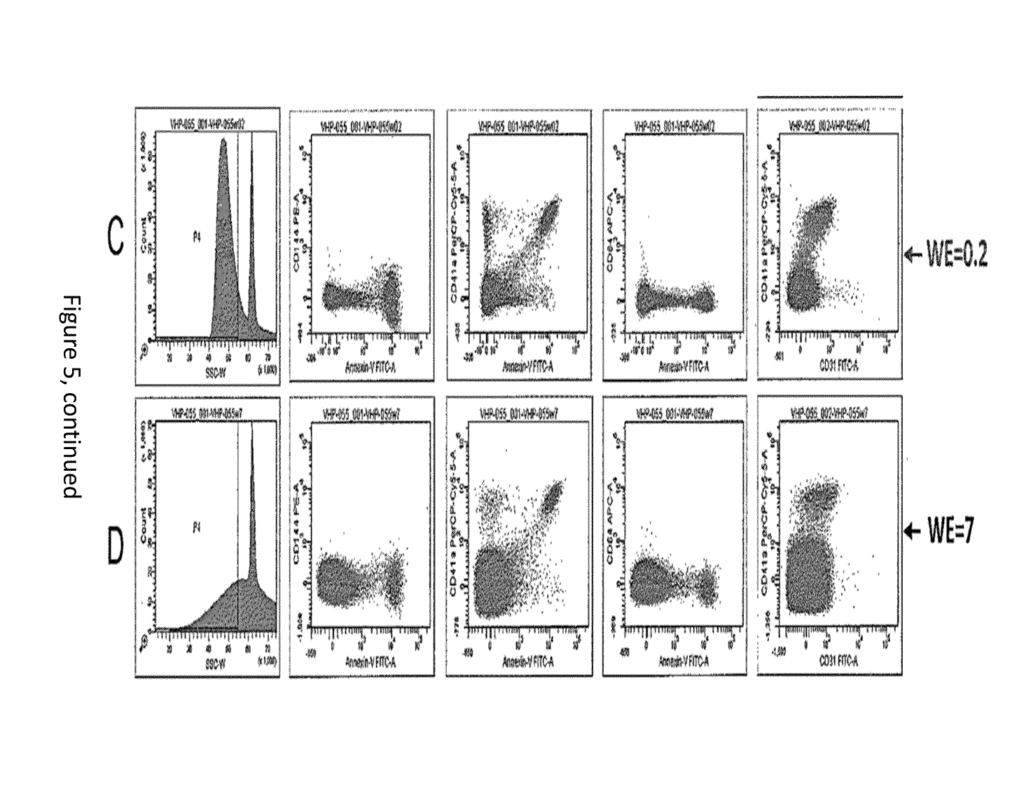

As illustrated in FIGS. 6, 0.3, 1 and 3 μm beads were used to estimate MPs size (A and B on Canto A and C on Gallios). MPs were gated on less than 1 μm (D gated on SSC-W histogram and E on FSC/SSC dot plot on Canto and F on Gallios). For the Canto A setting, forward and side scatter thresholds were set to 5000 and 200, respectively, and the window extension was set to 0.2. For the Gallios setting, the discriminator value for FS was set to 1 and the Forward Scatter Collection Angle was W2.

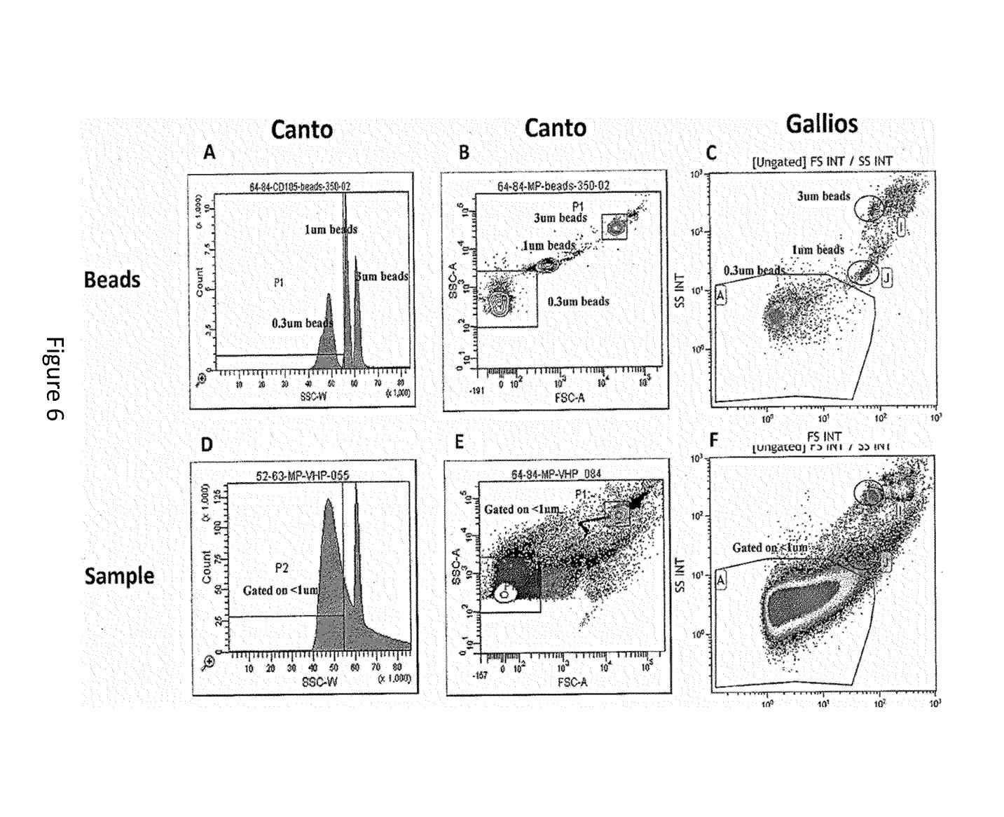

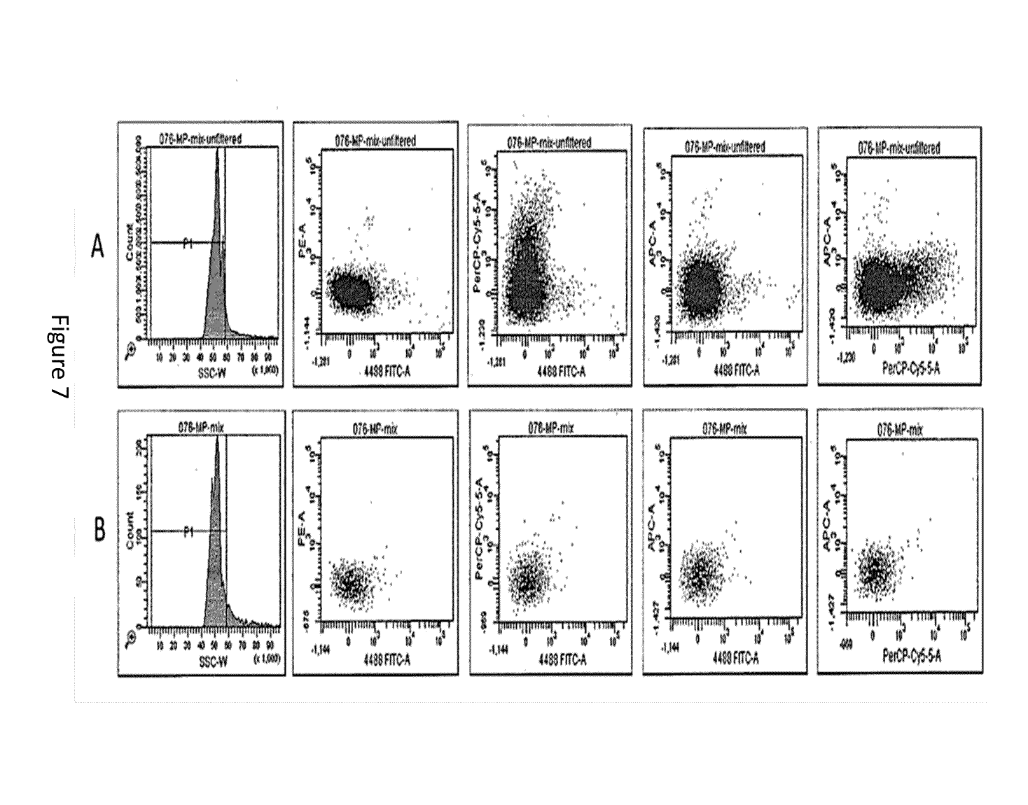

As illustrated in FIG. 7, the same volume of antibodies used for MP detection in 500 μl of double filtered PBS was run on Canto A. Unfiltered antibodies (Row A) showed more false positive events than double-filtered antibodies (Row B). All reagents used for MPs detection should be double-filtered through 0.1-0.22 μm low protein binding filter to remove the antibody aggregates and background noise from running buffer.

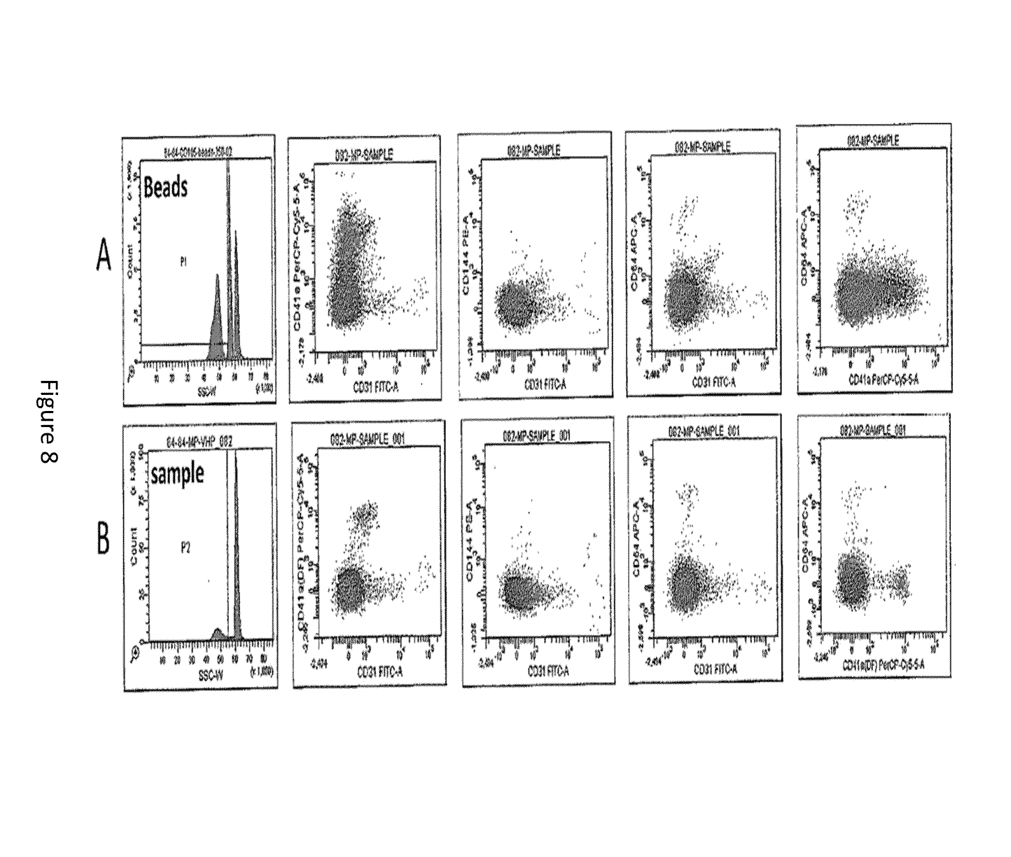

As illustrated in FIG. 8, 50 μl of PFP were labeled with unfiltered antibodies (Rows A and C) and double-filtered antibodies (Rows B and D). Rows A and B were gated on less than 1 μm on SSC-W histogram plot. Rows C and D were gated on less than 1 μm on FSC and SSC dot plot. Pre-filtering antibodies helps to reduce the false positive particles by removing the aggregation of antibodies.

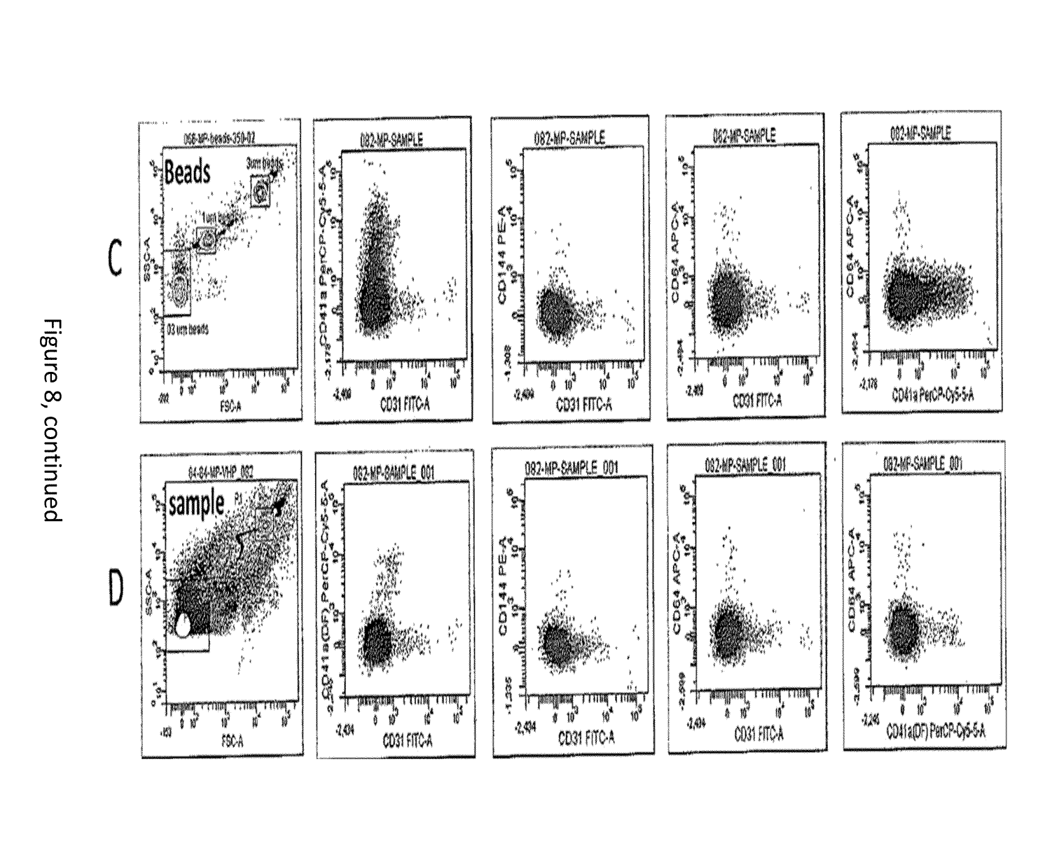

As illustrated in FIG. 9, MPs were detected on both Canto and Gallios. MPs were gated on less than 1 μm on both Canto (Row A) and Gallios (Row B). Positive MPs were determined based on fluorescence minutes one (FMO) tubes.

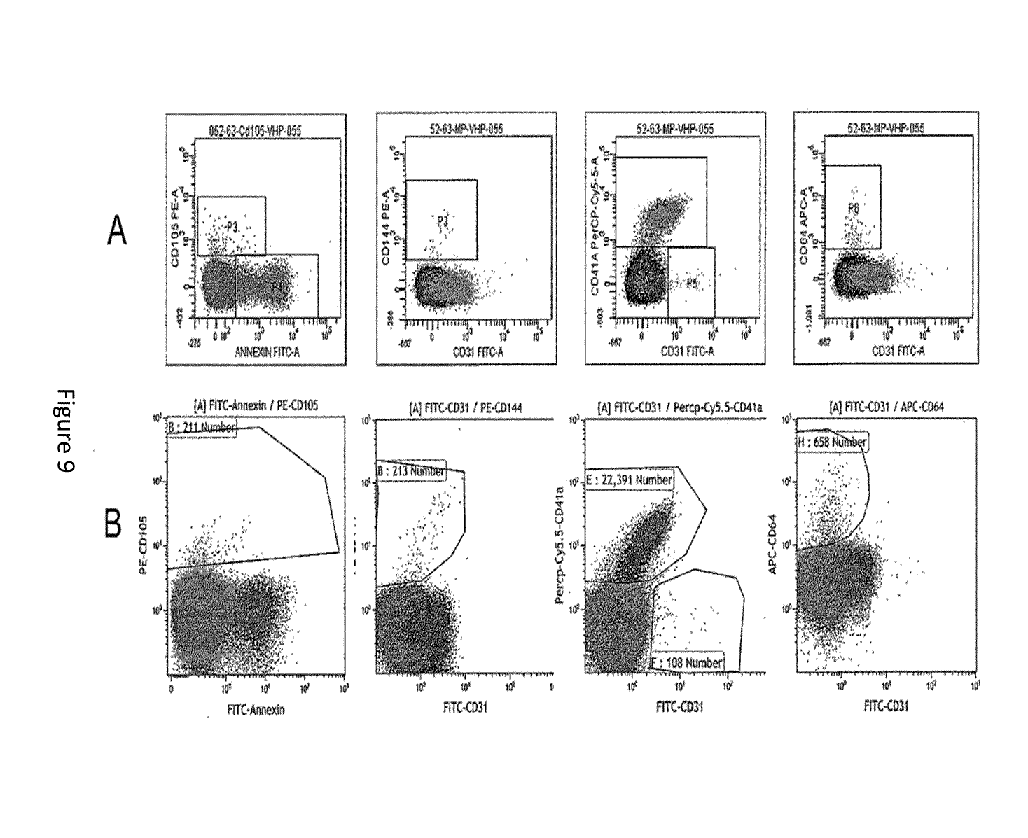

As illustrated in FIG. 10, computed Spearman rank correlations were used to compare the MP counts between the two platforms, most correlations exceeded 0.8 with the exception of CD105(+) which demonstrated a correlation of 0.6 (P<0.05). MPs counts on Gallios were two times greater than on Canto A. Row A shows the correlations of the two platforms, while Row B shows the comparison of MPs number on the two platforms.

As demonstrated in this Example, optimization of the cytometer setup can significantly diminish background noise. Preferably, the reagents, including buffer and antibodies, should be double-filtered by 0.1-0.2 μm low protein binding filter to reduce the aggregation of antibodies and background noise from the buffer. The values obtained on the Gallios and FACSCanto were correlative, and the Gallios detected larger numbers of events in the region of interest.

### Example 2

### Relationship of Microparticles to Progenitor Cells as a Measure of Vascular Health in a Diabetic Population

The relationship of MPs to PCs/PACs was examined to see if it could be used as an improved and clinically feasible index of vascular pathology. Plasma samples were collected from patients with early-stage (ES, Diagnosis <1 year) and long-term (LT, Diagnosis >5 years,) Type 2 diabetes and compared with age related healthy subjects (H). PC and MP subtypes were measured by a combination of flow cytometry and ELISA-based methods. The ratio of procoagulant MPs/CD341 PCs proved a valuable index to distinguish between subject groups (P5 0.01). This index of compromised vascular function was highest in the LT group despite intensive statin therapy and was more informative than a range of soluble protein biomarkers.

Unexpectedly, a relationship was found between MPs and PCs in Type 2 diabetes. This ratio provides a quantitative and clinically feasible measurement of vascular dysfunction and cardiovascular risk in patients with diabetes.

The following materials and methods were used in Example 2:

### Patient Recruitment and Study Design

Patients diagnosed with Type 2 diabetes within the preceding 12 months were termed “Early Stage” (ES), and those diagnosed more than five years ago were termed “Long Term” (LT). Age related “Healthy” (H) subjects were recruited into the study on the basis of having no prior or current history of diabetic- or cardiovascular-related conditions and were not taking any type of CV-related medication including statins or medication for hyperlipidemia, hypertension or diabetes. Four (36%) of the ES group and 14 (67%) of the LT group were receiving statin medication, along with a range of other medications to treat diabetes, hypertension, and other conditions. Regarding family history of cardiovascular disease, 12 of the 18 (66%) subjects in the healthy group, 10 of the 11 (90%) in the early stage and every participant (100%) in the long term group responded that a prior family history of cardiovascular disease existed. The average length of diabetes in the LT patients was 20 years. All study participants were nonsmokers. Written informed consent was obtained from all study participants and study protocols were approved by the Institutional Review Board (IRB). Each subject donated about 50 mL of venous blood at around 8:00 am in the morning and all subjects had fasted the night beforehand. Blood was collected in heparinized (Baxter) syringes for cell and soluble protein analysis (30 mL), SodiumCitrate for microparticles (10 mL) and EDTA for lipid analysis (10 mL). Demographic data, medical/medication history, physical examination, and vital signs were recorded for each subject.

### Flow Cytometry for PCs/PACs

Less than 1 h post sample collection, white blood cells were isolated from 30 mL of blood using ammonium chloride lysis as previously described. Platelet counts were not determined Cell staining, gating strategy, flow cytometric methods, and analysis were followed as described. Approximately 5E6 cells were stained with a 6-color antibody panel: FITC-antiCD31 (PECAM) (Pharmingen), PE-anti-CDI33 (Miltenyi Biotec.), PerCP-Cy5.5-anti-CD3,-CDI9,-CD33 (Becton Dickinson), APC-H7 anti-CD45 (Becton Dickinson), PE-Cy7-anti-CD34 (Becton Dickinson), and APC-anti-VEGF-R2 (R&D Systems). Viability was assessed by propidium iodide exclusion. Using a Becton-Dickinson LSRII cytometer, 2E6 live events were processed for each sample and the six fluorescent markers along with light scatter allowed only viable, low to medium side scatter. Singlets that were CD3,19,33-negative were analyzed for PCs and PACs. Singlets were gated as the prominent cluster of cells identified from a plot of side scatter width versus forward scatter width to ensure that cell aggregates were excluded from analysis. Fluorescence minus one (FMO) samples was used as negative controls. Cell populations (PCs and PACs) were quantified as a percentage of mononuclear cells calculated as number per ml blood. Analysis focused on subset definitions for PCs and PACs (Table 1). Data analysis was performed using FlowJo analytical software (Treestar, Ashland, Oreg.).

TABLE 1
Cell or Particle Genotype Per Surface Marker by Flow Cytometry

| Cell/Particle Type | Marker definition |
|---|---|
| Progenitor cell (PC) | CD133⁺, CD34⁺, CD133⁺/34⁺ |
| Proangiogenic | CD133⁺ VEGF-R2⁺, |
| cell (PAC) | CD34⁺ VEGF-R2⁺, |
| | CD133⁺ CD34⁺ VEGF-R2⁺ |
| Endothelial | CD144⁺ |
| microparticle (EMP) | |
| Platelet | CD41⁺ |
| microparticle (PMP) | |
| Monocyte | CD14⁺ |
| microparticles (MMP) | |
| Phosphatidylserine⁺ | AnnV⁺ |
| (PS⁺) MP | |

### MP Isolation

Platelet-poor plasma (PPP) was obtained from citrated blood within an hour after blood collection in order to isolate MPs. Whole blood was centrifuged at 1500 g for 15 min, supernatant collected, and PPP obtained by centrifugation at 13,500 g for 5 min at room temperature. For each subject, PPP was aliquoted into separate tubes and stored at −80° C. until subsequent use. All samples used were subjected to only one freeze-thaw cycle.

### Flow Cytometry for MPs

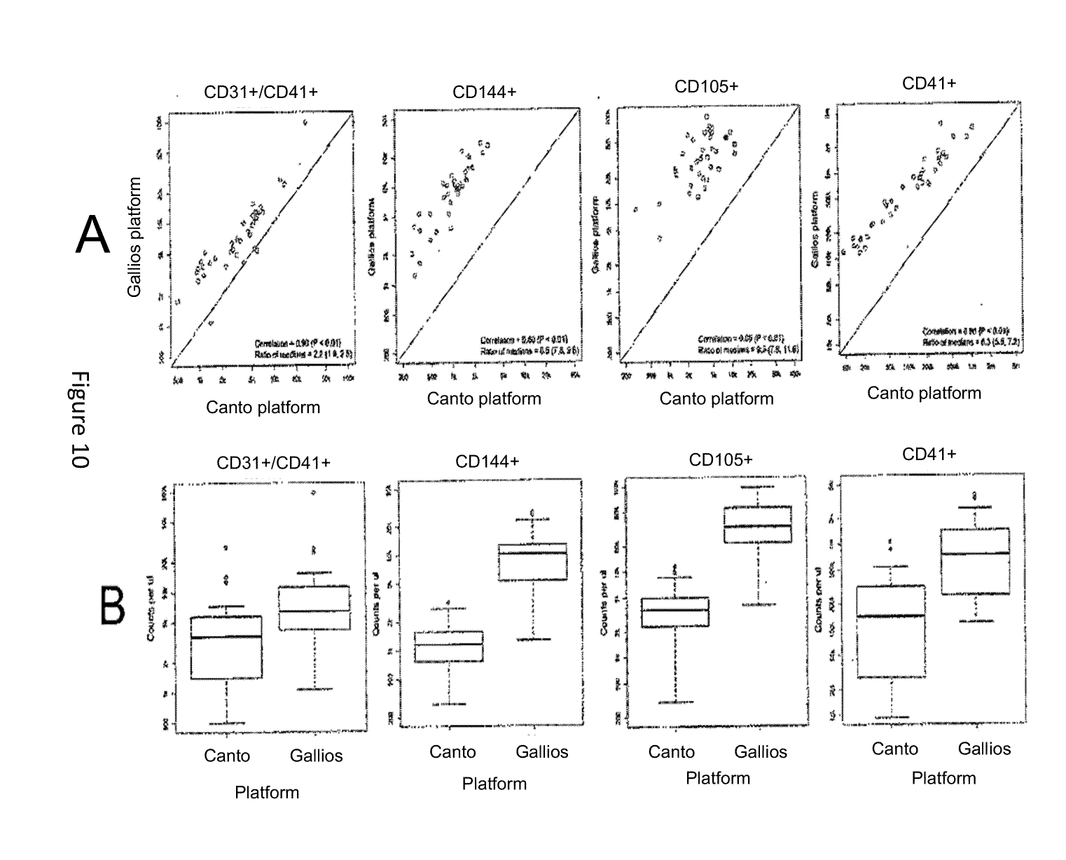

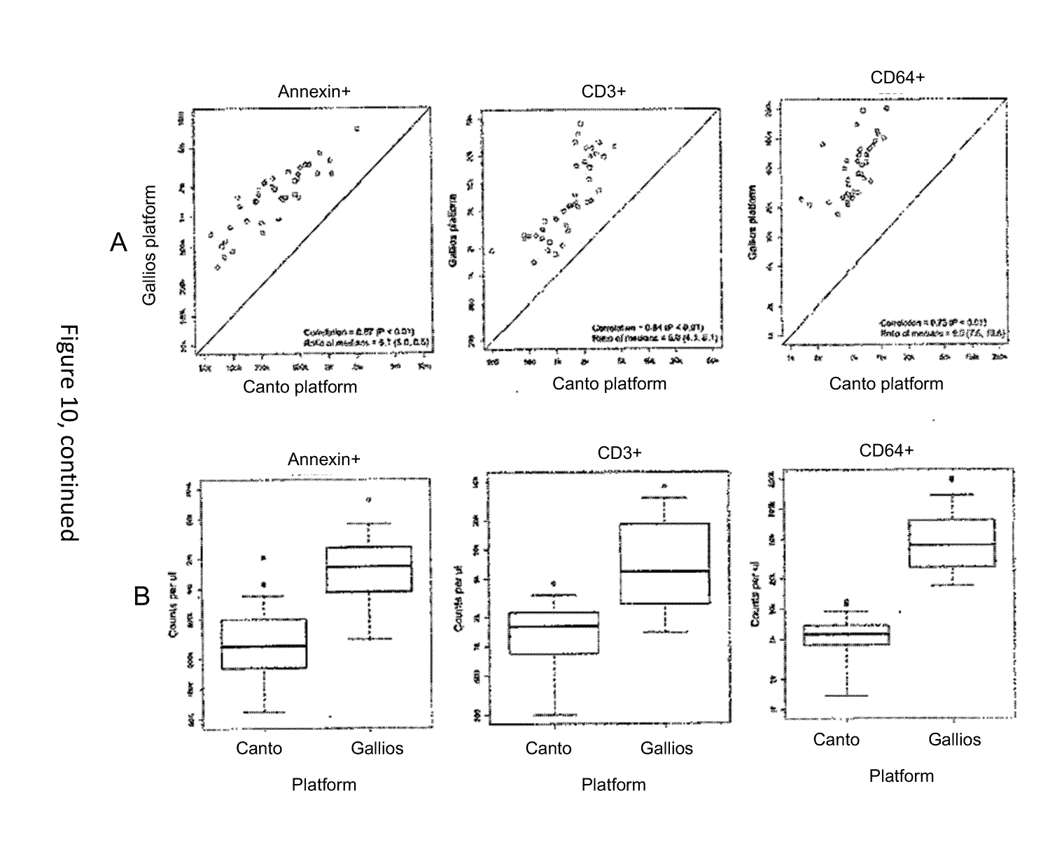

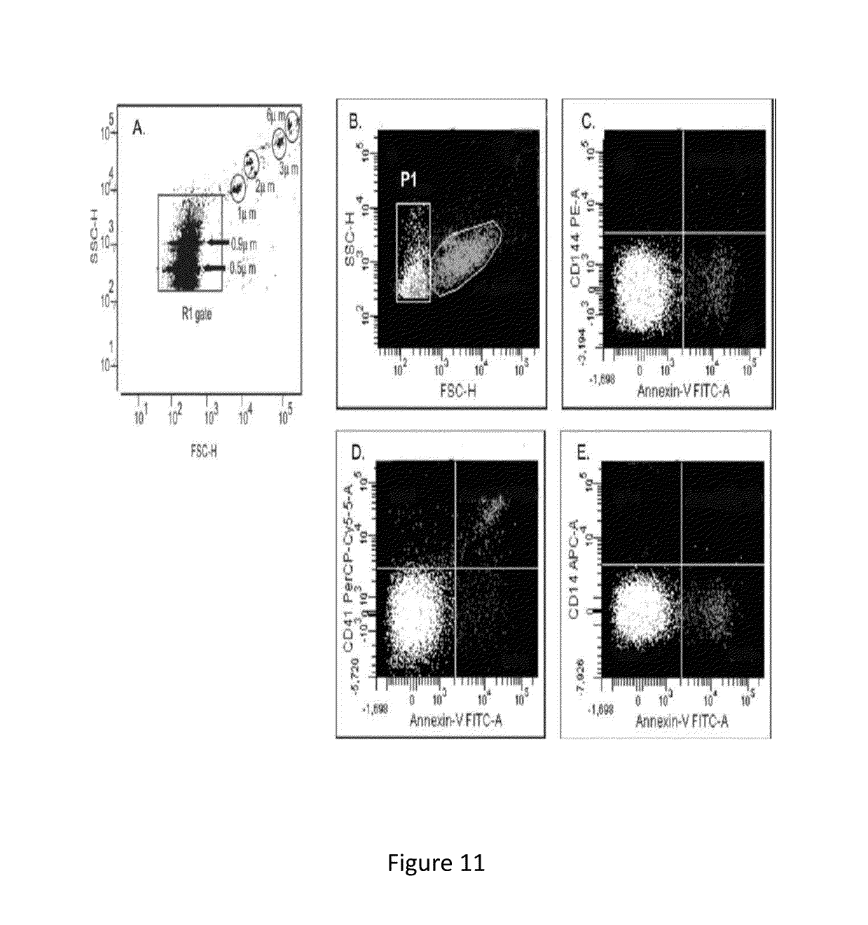

For characterization and quantification of MPs, PPP was incubated with a mixture of Annexin-V (FITC), PECYS-CD41a, APC-CD14 (BD Biosciences) and PE-CDI44 (R&D System) in 1×BD annexin-V binding buffer (10 mM Hepes, pH 7.4, 140 mM NaCl, and 2.5 mM CaCl2) (BD Biosciences) for 30 min at RT in darkness, then 1× BD annexin-V binding buffer was added to make total volume of 1 mL/tube. The negative control was prepared as PPP stained with Annexin-V (FITC) and same amount of matched isotype control antibodies in calcium-free binding buffer. Using a BD Biosciences FACSCanto cytometer, a P1 region (<1 lm) on FSC-H and SSC-H scatter (log scale) was defined by calibrator beads (FIG. 11A). The number of MPs per 1 L was determined using the P1 region and also 6-1 m microsphere beads (Bacteria Counting Kit, Invitrogen) to determine volume of sample (1 L) analyzed (FIG. 11B). The number of MPs stained with each specific Ab and AnnexinV was analyzed and determined using FACSDiva software (BD Biosciences) and expressed as MPs/1 L. Characterization of cellular origin of MPs by positive antibody staining is listed in Table 1, above.

### MP Plate-Based Capture Assay

Concentrations of PS+ MPs were determined using the Zymuphen MP Activity Kit (Aniara-CAT#A521096). In this assay, MPs were captured from PPP onto insolubilized annexin-V and their PS content was measured by a functional prothrombinase assay. This offers an indirect measurement of total MP procoagulant activity via measurement of nM of phosphatidylserine on the outer membrane surface. MPs measured by this method are expressed in nM of phosphatidylserine equivalent.

### Soluble Proteins

Soluble proteins were determined from citrated plasma by electro-chemiluminescant detection using commercially tested kits, as per the manufacturer's instructions. Meso-Scale Discovery multiplex kits, including the Vascular Injury II assay kit (CAT # K11136C-1) was used to measure SAA, CRP, VCAM1, and ICAM1. IL6, IL8, TNFa, and IL1b were measured using the Human Pro-Inflammatory Base Kit (KI5025A-5). The Human Hypoxia Assay (CAT# KI5122C-1) measured VEGF, IGFBF-1 and EPO. Plasminogen activator inhibitor (PAI-1) was detected with the Imubind kit from AmericanDiagnostica. Stromal cell-derived factor 1 (SDF-1) was measured by R&D systems (CAT# DY350). IL1b, SAA, IL6, and IL8 were below detection with these assays.

### HbA1c

HbA1c was performed using the Primus boronate affinity HPLC method (Primus Corporation, Kansas City, Mo.) according to the manufacture's protocol.

### Lipid Profile Analysis

Blood samples were collected in EDTA for lipid analysis. HDL was performed using an enzymatic in-vitro assay for the direct quantitative determination of human HDL cholesterol on Roche automated clinical chemistry analyzers following the manufacture's protocol. Triglyceride and cholesterol were performed using VITROS TRIG slides and VITROS chemistry products calibrator kit 2 on VITROS chemistry systems (VITROS 950 Chemistry System). LDL was calculated by the Friedewald Equation.

### Complete Blood Count (CBC) and White Blood Count (WBC) Differentiation Analysis

Blood cell analysis was performed using COULTER LH 780 Hematology Analyzer (Beckman Coulter) following the manufacture's protocol.

### Statistical Analysis

All univariate comparisons of the disease state groups used two-sided non-parametric tests or Chi-squared comparisons of proportions, which did not require Gaussian distributions. Most of the measured characteristics, especially the MP, PACs, and soluble protein levels had strongly non-Gaussian distributions. The disease state group comparisons of the subject characteristics did not assume disease state related trends in the responses. These comparisons used the KruskalWallis (KW) test (nonparametric ANOVA comparisons). The analyses of the PC, PAC, MP, and soluble protein levels assumed trends in the responses relative to the disease state. The assessment of the statistical significance of these trends used the Jonckheere-Terpstra (JT) test (nonparametric trend comparisons). The post-hoc comparisons of differences between individual disease state groups (e.g. ES versus LT) used the Wilcoxon two sample test. The comparisons of the proportions of patients by gender applied the exact Chi-square test of the equality of proportions across the groups. This test compared the observed proportions in the disease state groups against the hypothesis that the proportions were the same for all the disease state groups. The other characteristic proportions (e.g. statin use) were criteria for exclusion from the H group, thus only the ES and LT groups were compared. The analysis of the MP and PAC relationships to the disease state after adjustment for specific covariates were based on logistical regression models with the covariates already included in the models.

On the basis of the assumption of a similar relationship of the disease state differences and the response variabilities, a power calculation was performed with data from Koga et al. using CD144+ EMPs. The sample size of 11 in ES, 22 in LT, and 18 in H, had about 90% power in detecting the relative disease state differences (control versus Diabetes Mellitus) as noted in the aforementioned study.

### Example 2

### Results

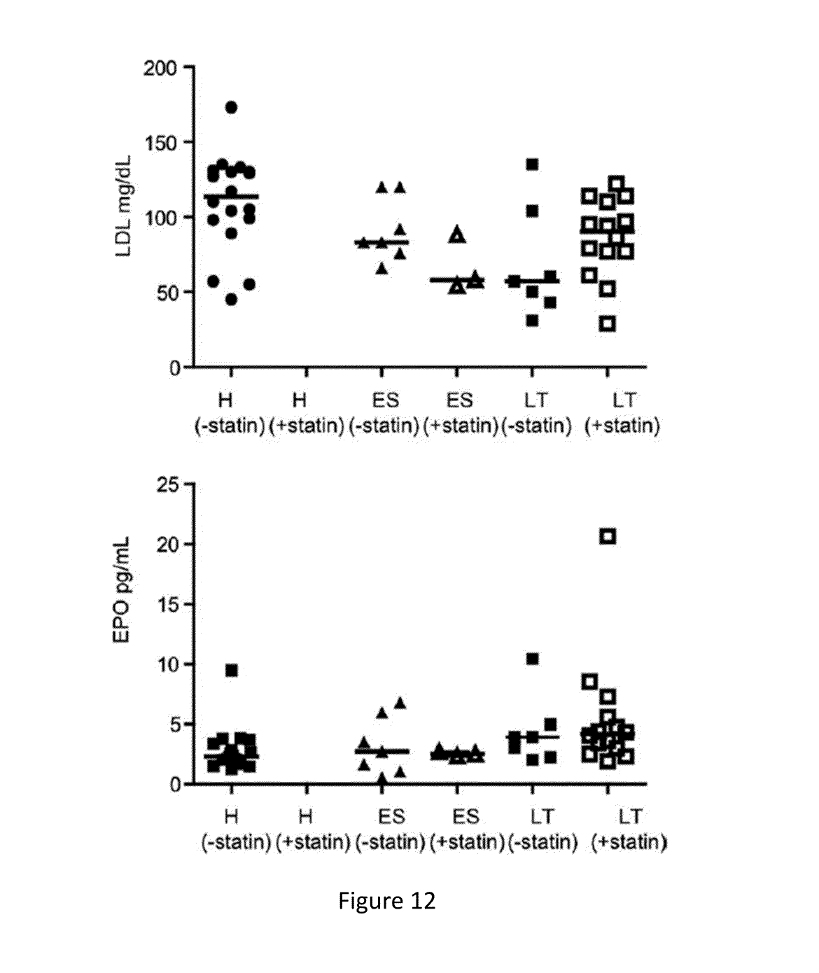

On the basis of previous findings that diabetes is a risk factor for cardiovascular disease and duration with diabetes is a further additive component of that risk, we recruited ES and LT patients. Subject characteristics are presented in Table 2. A total of 41 subjects (mean age 57 years) were included. Age and gender did not differ between the three groups. As anticipated, the standard marker of blood glucose control, glycated hemoglobin, HbA1c, was significantly altered between all diabetic and healthy individuals, but could not discriminate between ES and LT groups. The lower levels of LDL observed in the diabetic groups was most likely related to statin use (36% in ES, 67% in LT). To investigate this effect in the diabetic groups, LDL levels were plotted in each group separated by statin use, as shown in FIG. 12A. LT diabetics receiving statins had reduced LDL levels compared to healthy individuals, indicating that without such therapy LDL levels in this cohort would have been substantially higher. The fact that LDL levels were lower in LT patients without statins versus with statins may be due to the small sample size of the former (n=7) compared with the latter (n=14). HDL and Triglycerides did not differ between the three groups. Although LDL levels were controlled in the LT group, systolic blood pressure was higher (P<0.01) in LT versus H groups. Importantly, as our studies investigated the role of certain cell derived markers (particularly monocyte MPs), the monocyte and lymphocyte counts were statistically unchanged among the three groups. Red blood cell count was not altered between ES and H groups but decreased significantly in the LT group. Similarly, hemoglobin levels were significantly lower in the LT versus H group (Table 2).

TABLE 2
Patient Characteristics

| | H (18) | | ES (11) | | LT (22) | | P |
|---|---|---|---|---|---|---|---|
| Age (y) | 57 ± | 14 | 51 ± | 13 | 64 ± | 13 | NS |
| Gender (% males) | 61% | | 64% | | 81% | | NS |
| Office systolic BP (mmHg) | 125 ± | 11 | 133 ± | 14 | 139 ± | 13^{†} | * |
| Office diastolic BP (mmHg) | 78 ± | 5 | 80 ± | 11 | 76 ± | 12 | NS |
| Total cholesterol (mg/dL) | 185 ± | 34 | 168 ± | 43 | 156 ± | 34^{†} | ** |
| LDL (mg/dL) | 109 ± | 32 | 84 ± | 23^{¶} | 80 ± | 31^{†} | ** |
| HDL (mg/dL) | 55 ± | 11 | 44 ± | 13 | 51 ± | 19 | NS |
| TG (mg/dL) | 107 ± | 61 | 169 ± | 129 | 126 ± | 103 | NS |
| HgbA1c | 5.3 ± | 0.3 | 7.5 ± | 1.7^{‡} | 7.1 ± | 1.5^{†} | ** |
| WBC | 5.3 ± | 1 | 6.2 ± | 2 | 5.8 ± | 1 | NS |
| Monocyte Count (n/μL) | 432 ± | 148 | 531 ± | 255 | 483 ± | 118 | NS |
| Lymphocyte Count (n/μL) | 1557 ± | 522 | 2074 ± | 920 | 1660 ± | 634 | NS |
| RBC (million/μL) | 4.48 ± | 0.45 | 4.55 ± | 0.36 | 3.99 ± | 0.54^{∥,#} | ** |
| Hemoglobin (g/dL) | 13.92 ± | 1.58 | 13.46 ± | 1.83 | 12.22 ± | 1.45^{∥} | ** |

| Hypertension | 0 | 7 | (64%)^{‡} | 15 | (68%)^{†} | NA |
|---|---|---|---|---|---|---|
| Hypercholesterolemia | 0 | 8 | (72%)^{‡} | 14 | (63%)^{†} | NA |
| Myocardial infarction/angina | 0 | 1 | (9%) | 2 | (9%) | NA |
| Stroke | 0 | 0 | | 1 | (4%) | NA |
| Peripheral artery disease | 0 | 1 | (9%) | 3 | (14%) | NA |
| Statin (% use) | 0 | 4 | (36%) | 14 | (67%) | NA |

Data are means ± SD.
*P < 0.01, **P < 0.0001, NS, nonsignificant; NA, not analyzed.
Post-hoc comparison significance levels (not adjusted for multiple comparisons).
^{†}P < 0.01 LT vs. H, ^{‡}P < 0.01 ES vs. H, ^{∥}P < 0.05 LT vs. H, ^{¶}P < 0.05 ES vs. H, ^{#}P < 0.05 LT vs. ES.
KW was used to test significance between all three groups for measured values.
For measured data, post-hoc analyses between patient groups used the two sample Wilcaxon test.
Comparisons for gender data used the two-sided exact Pearson Chi Square test for comparisons of the three groups and for the post-hoc analyses.
The characteristics with zero healthy patients had that as a group requirement, thus comparisons to that group were not analyzed.

Assessment of PCs, PACs, and total MPs along with a range of cell-specific MPs was performed. A general relationship was observed in which PC and PAC levels decreased, and MPs and most MP subtypes increased with either onset or disease duration (Table 3). The exceptions to the above was that PCs increased from H to the ES state, but all fell below H levels in the LT disease state, however this was not statistically significant (P<0.05). PACs were detected at significantly lower frequencies than PCs and some significant changes were observed.

All three PACs were approximately equivalent between H and ES disease state, but CD133VEGF-R2 dropped significantly from ES to LT subjects. Significant reductions in triple positive PACs, CD133+, CD34+, VEGFR2 were only observed between H and LT groups. It is interesting to note that the number of individuals within each group with undetectable levels of triple positive PACs (CD 133+, CD34+, CDVEGF-R2) by flow cytometry also rose dramatically with onset of disease (33% in H, 63% in ES, and 68% in LT). For cell-derived MPs, levels of circulating EMPs and PMPs were altered significantly between H and LT (P=0.03). CD 14 monocyte derived MPs were neither affected by disease state or duration (Table 3). Changes in AnnV+ MPs measured by flow cytometry and nM of PS+ MPs measured by the plate based assay, were statistically equivalent (P=0.02). However, the flow cytometry method could detect differences with onset of disease, H vs. ES, whereas the plate-based method detected changes with disease duration, ES and LT. The differing resolution between the two assays may be due to their separate readouts; cytometry (AnnV+ MPs) counts the number of PS+ MPs whereas the plate captures PS+ MPs and quantifies using a prothrombinase assay. Nonetheless, both assays detected differences between the H and LT. These results demonstrate the possibility of employing a more feasible plate based assay to measure MPs in clinical studies than flow cytometry. A covariate adjustment was also performed to assess if the observed group differences remained significant after the covariates, age, gender, hypertension, and statin use were included in the regression model. As hypertension and statin use were exclusion criteria within the H group, only age and gender could be used in those comparisons. With adjustment for age and gender, AnnV+ MPs was still significant (P=0.02) between H and ES as were EMPs (P=0.03) and PMPs (P=0.01) between H and LT. AnnV+ MPs were borderline significant (P=0.053) between H and LT. For comparisons between ES and LT, adjusting for age, gender, hypertension, and statin use, no other covariate improves the model after age is included.

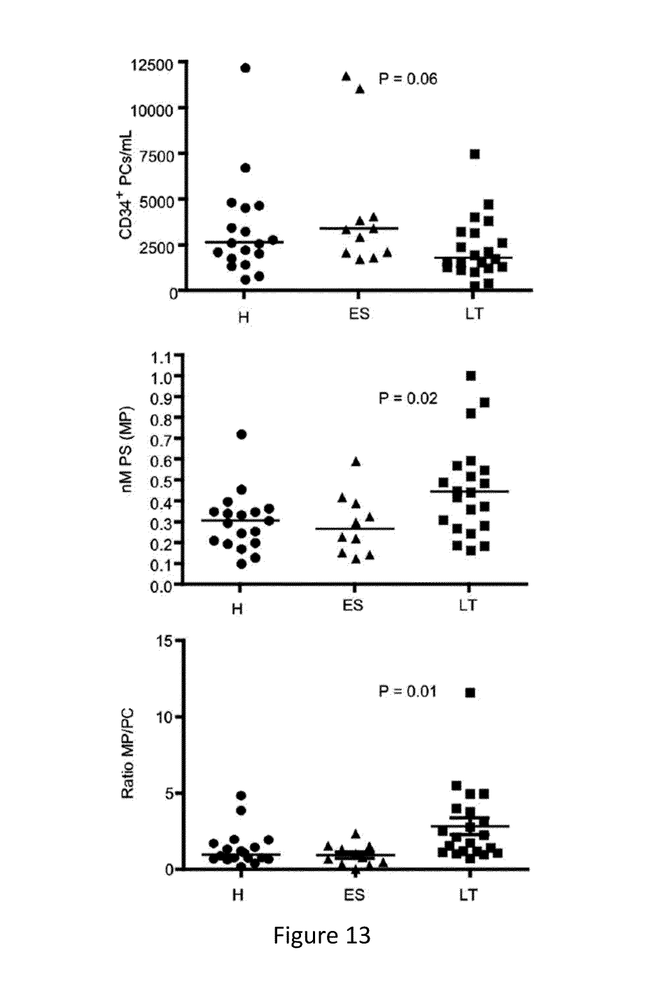

The measurement of double and triple positive PACs and of cell derived MPs, by flow cytometry, is both technically challenging and expensive. The value of measuring single positive PCs by flow cytometry and PS+ MPs by the plate based assay was investigated. CD34+ PCs displayed borderline significant reductions from H to ES (P=0.06) whilst the measurement of procoagulant MPs by the plate-based assay displayed a significant upward trend with (P=0.02), as shown in FIG. 13.

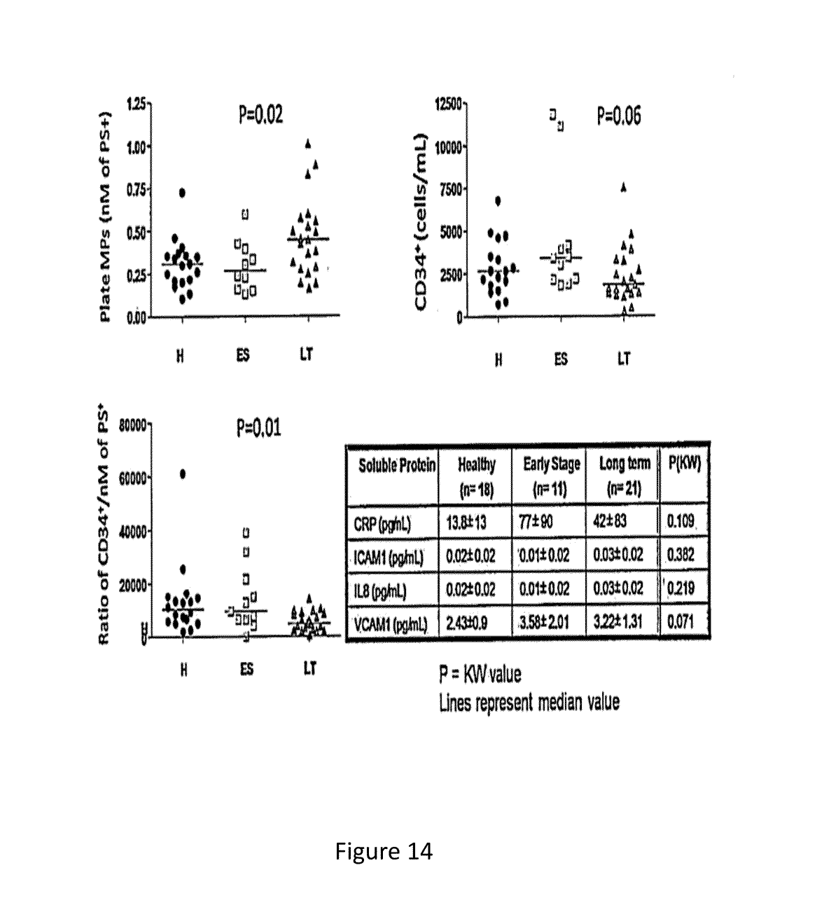

It was assessed whether the ratio of MP/PC offered additional information over the investigation of PCs/PACs or MPs alone. Interestingly, the ratio of PS+ MPs/CD34+ PC proved a valuable index to distinguish the subject groups (P=0.01) (FIG. 13) and this change was more significant than any of the other single PC, PAC, or MP subtypes analyzed (Table 3). The median levels of ELISA plate MPs, flow cytometry measured CD34 cells, the ratio between the two levels, and a comparison with “non cell” atherosclerotic biomarkers are shown in FIG. 14. PCs, PACs, and MPs were also compared to a range of soluble protein markers. The MP/PC ratio was more predictive than a number of often utilized soluble proteins including CRP, ICAM1, and VCAM1 (Table 4). These soluble proteins were quantified via multiplex assays, a cost effective and efficient method often employed in clinical studies. TNFa, VEGF, IGFBF-1, SDF-1, and PAI-1 although detectable were not significant. Of interest, EPO concentration was higher in the LT group (P ¼0.004), (Table 4) and was not due to exogenous recombinant human EPO (rhEPO) or linked to statin use (FIG. 12B). Those within the LT group had significantly depressed red blood cell (RBC) count and hemoglobin and were therefore approaching an anemic state in comparison to both Healthy and ES (Table 2).

TABLE 3
Cell and Cell Derived Biomarkers

| Marker type | H | | ES | | LT | | P |
|---|---|---|---|---|---|---|---|
| CD133⁺ PC | 954 | (712-157) | 1052 | (538-1277) | 639 | (448-1243) | 0.08 |
| CD34⁺ PC | 2676 | (1763-4644) | 3432 | (2153-4125) | 1818 | (1341-3268) | 0.06 |
| CD133⁺ CD34⁺ PC | 778 | (618-1251) | 868 | (407-1228) | 605 | (356-1208) | 0.15 |
| CD133⁺ VEGF-R2⁺ PAC | 8.6 | (0-20) | 18.3 | (0-21) | 0 | (0-9) ^{#} | 0.04* |
| CD34⁺ VEGF-R2⁺ PAC | 15.3 | (9-26) | 7.9 | (6-40) | 8.0 | (0-18) ^{#} | 0.03* |
| CD133⁺ CD34⁺ VEGF-R2⁺ PAC | 6.9 | (0-12) | 0 | (0-20) | 0 | (0-7)^{∥} | 0.04* |
| CD144⁺ MPs | 204 | (93-411) | 453 | (187-616) | 571 | (207-892)^{∥} | 0.03* |
| CD41⁺ MPs | 300 | (155-583) | 693 | (239-894) | 367 | (258-939)^{∥} | 0.03* |
| CD14⁺ MPs | 120 | (68-347) | 340 | (178-469) | 230 | (77-488) | 0.15 |
| AnnV⁺ MPs | 355 | (240-760) | 761 | (449-996)^{¶} | 682 | (330-1044)^{∥} | 0.02* |
| nM of PS⁺ MPs | 0.31 | (0.21-0.36) | 0.27 | (0.16-0.40) | 0.45 | (0.28-0.56)^{∥,#} | 0.02* |

Data are medians (25%-75% interquartile range).
P(A)C values correspond to Cells/mL, MP values correspond to MPs/μL exceptPS⁺ MPs which corresponds to nM of PS equivalent by plate-based assay.
H, healthy; ES, early stage diabetes; LT, long-term diabetes.
*P < 0.05. Post-hoc comparison significance levels (not adjusted for multiple comparisons).
^{∥}P < 0.05 LT vs. H, ^{¶}P < 0.05 ES vs. H, ^{#} P < 0.05 LT vs. ES.
P calculated by JT test to test for differences between the three groups.
Post hoc analyses between patient groups used the two sample Wilcoxon test.

TABLE 4
Soluble Protein Analysis

| | H | ES | LT | P |
|---|---|---|---|---|
| EPO (pg/mL) | 2.8 ± 1.8 | 3.0 ± 2 | 5.1 ± 4.2^{†} | 0.004** |
| CRP (pg/mL) | 13.8 ± 13 | 77 ± 90^{¶} | 42 ± 83 | 0.109 |
| ICAM1 (pg/mL) | 1.4 ± 0.5 | 2.7 ± 1.8^{¶} | 1.6 ± 0.1^{#} | 0.382 |
| VCAM1 (pg/mL) | 2.43 ± 0.9 | 3.58 ± 2.01 | 3.22 ± 1.31 | 0.071 |

**P < 0.01 H, healthy; ES, early stage diabetes; LT, long-term diabetes.
Post-hoc comparison significance levels (not adjusted for multiple comparisons).
^{†}P < 0.01 LT vs. H, ^{¶}P < 0.05 ES vs. H, ^{#}P < 0.05 LT vs. ES.
P calculated by JT test to test for differences between the three groups.
Post-hoc analyses between patient groups used the two sample Wilcoxon test.

It is believed this is the first study showing PACs decrease and MP increase with both onset and duration of Type 2 diabetes. Additionally, evidence is provided for the value of assessing the ratio of MPs to PCs (nM of PS+ MPs/CD34+ PCs), a measurement that is clinically feasible and more informative than some standard protein markers. PCs and MPs are not byproducts of cardiovascular disease but active components of the disease, and therefore reflect specific disease pathways. For instance, MPs are not only markers of cellular damage but also active agents in promoting endothelial dysfunction and coagulation. A reduction in PCs and PACs indicates a loss of vascular reparative ability. In demonstrating that levels of both PCs/PACs and MPs correlate with duration of diabetic disease, our results also provide mechanistic insights into the stepwise etiology of diabetes and its contribution to vascular pathology.

The presence of coronary artery disease (CAD) is often asymptomatic in individuals with diabetes. Although recent noninvasive studies indicate that CAD can be detected in significant numbers of these individuals, a routine screening approach has not been shown to be clinically useful or cost effective. Biomarkers, and particularly cell-derived biomarkers, may prove to be more predictive of cardiovascular events. The search for cell biomarkers of patients at risk for vascular complications is promising but has not become a part of clinical practice because a rapid, easy to perform cell based assay has not been validated.

As demonstrated in Example 1, significant alterations in both PCs/PACs and cell derived MPs in Type 2 diabetes and also with duration of disease were identified. This is illustrative of the value of using the ratio of MPs/PCs, which encompasses two biologically relevant markers that impact functionally on disease progression. Further, it shows that this ratio may be more informative than many individual standard protein biomarkers commonly used to stratify individuals at heightened cardiovascular risk. From a clinical standpoint, the results from these studies indicate that a single platform high throughput, multiplexed flow cytometry assay for hematopoietic progenitors and plate-based assay for MPs is a feasible and cost effective method to identify those individuals at highest risk for cardiovascular events.

### Example 3

### Study of MPs and PCs in Diabetes Mellitus (DM)

A study of 50 subjects was performed to evaluate the level of MPs and PCs in patients with recently diagnosed DM and those with long term diagnosis compared to healthy controls using flow cytometry and ELISA. In addition to measuring endothelial, platelet and monocyte MPs, endothelial progenitor cells (EPCs) were also measured.

The following materials and methods were used in Example 3:

### Study Population and Methodologies

Patients diagnosed with Type 2 DM within the preceding 12 months were termed ‘Early Stage” (ES), and those diagnosed over 5 years were termed “Long Term” (LT). Age related “Healthy” (H) subjects were recruited into the study on the basis of having no prior or current history of diabetic or cardiovascular disease and were not taking any type of CV related medication, including statins. Only non-smokers were permitted to participate in the study. Each subject donated approximately 50 mL of venous blood. All subjects fasted the night before the visit and blood samples were drawn at approximately 8 am in the morning. Blood was collected in Heparinized (Baxter) syringe for PCs and soluble protein analysis (30 ml), Na-Citrate for MPs (10 ml) and EDTA for lipid analysis (10 ml). The MPs were measured using flow cytometry and also with a non cell specific ELISA assay that utilized annexin V antibody.

Data generated was determined to have non-Gaussian distributions and therefore two types of appropriate comparison tests were made. The difference across subject groups was determined using the Kruskal-Wallis (KW) test (non-parametric ANOVA comparisons). As there was a component of time (i.e. duration of disease) and potential trends across the subject groups, the Jonckheere-Terpstra (IT) test was employed, a non parametric test to compare trends in the data. As anticipated, the standard marker of blood glucose control, glycated hemoglobin, HbAlc, was altered significantly with disease duration (P<0.0001).

### Study Characterization of PCs and MPs

Cell derived MPs and PCs were identified and quantified in each subject group according to cell surface markers. Using flow cytometry, CD133 and/or CD34 expression defined PCs, whereas the additional surface expression of VEGF-R2 defined EPCs. MPs, initially defined by size (<1 um using flow cytometry) from platelet free plasma (PFP) were further characterized by cell type using single marker surface expression. Endothelial, platelet and monocyte MPs were determined by surface expression of CD144, 41 and 14 respectively. The anionic phopholipid, phosphatidylserine, detected on the outer leaflet of the plasma membrane on many cell derived MPs was determined by AnnV binding via flow cytometry. Additionally, a commercially available ELISA based plate based assay was utilized for MP detection. This offers an indirect measurement of total MP quantity via measurement of nM of phosphatidylserine on the outer membrane surface.

Specificities of monoclonal antibodies used in the identification of
microparticles

| Subtype | Antigen | Comment | References |
|---|---|---|---|
| Endothelial | CD144 | VE Cadherin | 1 |
| Platelet | CD41 | Gplibilla | 2 |
| Monocyte | CD14 | Endotoxin | 3 |
| | | Receptor | |

### Example 3

### Results

In general, PC and EPC levels decreased, and MPs and most MP subtypes increased with duration of disease in diabetic subjects (FIG. 11). Interestingly, the changes in EPCs were more pronounced than those of the progenitor cells. For cell specific MPs, levels of circulating endothelial MPs (EMPs) and platelet derived microparticles (PMPs) were altered most significantly between groups (FIG. 11). CD 14+ monocyte derived MPs (MMPs) were least affected by disease or duration. AnnV+ MP quantitation by flow cytometry and the ELISA plate based assay, displayed the same trend and significance between the select disease groups, providing validation and assurance for the use of this ELISA based assay in clinical studies. The number of individuals within each group with undetectable levels of triple positive EPCs by flow cytometry, increased with disease and disease duration, 33% in H, 63% in ES and 68% in LT. To identify what groups were most separated from each other, separate t-tests were performed of all the listed PCs, EPCs and MPs on the three combinations (i.e. Healthy vs EST, Healthy vs LTD and ESD vs LTD). Of these, H and LTD were most distinct from each other.

The results demonstrate that MPs are elevated and EPCs are lower in long term diabetics compared to healthy individuals. The ratio of CD34 positive cells compared to MPs was more robust than either alone in long term diabetics.

### Example 4

### Cytometric Fingerprinting

Cytometric Fingerprinting (CF) expresses the multivariate probability distribution functions corresponding to list-mode data as a “flattened”, computationally-efficient fingerprint representation that facilitates quantitative comparisons of samples. In order to test sensitivity and specificity, experimental and synthetic data were generated to act as reference sets for evaluating CF. Without the introduction of prior knowledge, CF was able to “discover” the location and concentration of spiked cells in un-gated analyses over a concentration range covering four orders of magnitude, to a lower limit on the order of 10 spiked events in a background of 100,000 events.

As demonstrated herein, this presents a new method for quantitative analysis of list-mode cytometric data. Also, a Bioconductor software package called “flowFP” has been developed to integrate fingerprinting approaches with other advanced methods of data analysis. This integrated facility creates a computational platform that supports both the generation and testing of hypotheses, eliminates sources of operator bias and provides an increased level of automation of data analysis. Importantly, fingerprint-based representation of flow cytometric data allows for direct fusion of data from multiple modalities, enabling an integrated analysis of dual-platform (flow cytometry and ELISA) data.

### Example 5

### Microparticle ELISA Assay

In some embodiments, the samples (e.g., platelet free plasma samples) tested by flow ctyometry can also be tested by Enzyme Linked Immunosorbent Assay (ELISA). ELISA samples can be serially diluted with 1% EDTA/saline. A pre-titrated amount (5 ug.ml in PBS) of Annexin V (BD Biosciences) can be added to each well of a 96-well microtiter plate as the capture reagent (to target the phosphatidylserine on MP surface) and incubated for 18 h at 4° C. Plates can be washed and PFP serially diluted samples can be added to the wells incubated for 18 h at 25° C. on a plate shaker (200 r.p.m.). After washing, pre-titrated amounts of biotinylated antibody (CD144,14,41a) can added to each well and incubated for 2 h at 25° C. on the plate shaker. Following washing, peroxidase-conjugated avidin can be added to each well. Each well may be subsequently washed and then incubated with peroxidase substrate solution for 20 min at room temperature. After this incubation, stop solution can be added to each well, and the absorbance can be measured with an EIA reader at a wavelength of 450 run.

Specifically, one can test this ELISA assay, and the single platform flow cytometry assay for specificity, accuracy, precision, linearity, limit and range as follows.

The specificity of the assay is the ability to assess unequivocally the binding of MP particles to annexin-V. The accuracy of the analytical procedure expresses the closeness of agreement between the value which is accepted either as a conventional true value or an accepted reference value and the value found. The ELISA assay is not a quantitative assay, while the flow cytometry assay is. Results on the two platforms can be compared. The precision of an analytical procedure expresses the closeness of agreement between a series of measurements obtained from multiple sampling of the same homogeneous sample under the prescribed conditions. Precision may be considered at three levels: repeatability, intermediate precision and reproducibility. Repeatability or intra-assay precision is most relevant to this study. The linearity of an analytical procedure is its ability to obtain the results which are directly proportional to the concentration of analyte in the sample. The detection limit of an individual analytical procedure is the lowest amount of analyte in a sample which can be detected but not necessarily quantified as an exact value. The range of an analytical procedure is the interval between the upper and lower concentration of analyte in the sample for which it has been demonstrated that the analytical procedure has a suitable level of precision, accuracy and linearity. The limit and range again can be assessed by comparing the data with the flow cytometry data.

### Example 6

### Study of MPs and EPCs in Patients with Diabetes Mellitus and Atherosclerosis

This study employed a novel method of scientific discovery based on a broad and comprehensive cell surface marker panel with an unbiased analysis scheme using cytometric fingerprinting to evaluate differences between the DM patients and HC. Unlike other studies, which only observe levels of either MPs or EPCs, by obtaining both MP and EPC samples, this study observed the balance of how vascular dysfunction, through the levels of MP, and reparative capacity, through the levels of EPC, interact.

### Patient Recruitment and Study Design

Patients diagnosed with type 2 DM for more than 5 years and with clinically apparent atherosclerosis, history of a myocardial infarction, stroke, claudication, or revascularization procedure were included in this study. Acute illness, myocardial infarction or stroke within 3 months prior to study enrollment and pregnant women were excluded from this study. Age-similar ‘healthy’ subjects were recruited into the study on the basis of having no prior or current history of diabetes or history of cardiovascular disease or major cardiovascular risk factors including smoking, hypertension or elevated LDL cholesterol.

Initially, 104 subjects were consecutively recruited (52 DM and 52 HC). Subsequently, due to a revision of the protocol requiring the analysis of fresh rather than frozen samples for MP analysis, additional subjects were consecutively recruited (n=14 DM and n=7 HC). From these subjects, 62 DM and 51 HC samples yielded data viable for quantifying EPCs. Forty-Eight DM and 48 HC samples were available for MP analysis. Data for 47 DM and 43 HC samples were available for a combined MP and EPC analysis. The Absolute Lymphocyte Count (ALC) from the complete blood count (CBC) results was used to normalize EPC counts, and five of the 96 samples did not have ALC and therefore were not available for the EPC analysis. Twenty-two subjects with EPC data did not have fresh samples available and therefore were not included in the MP analysis. Demographic information, medical and medication history, physical examination, and vital signs were recorded for each subject. Data was analyzed using the R environment for statistical computing (version 2.13.1, R Development Core Team, Vienna, Austria) and the flowFP (Rogers et al., 2008, Cytometry Part A 73A:430-441), flowCore (Ellis, B., Haaland, P., Hahne, F., Le Meur, N., and Gopalakrishnan, N. 2009. flowCore: Basic Structures for flow cytometry data. Bioconductor package version 1.18.0. Software may be obtained from http://bioconductor.org/packages/2.10/bioc/html/flowCore.html), and KernSmooth (Wand, M. (R port by Brian Ripley). 2011. KernSmooth: Functions for kernel smoothing for Wand & Jones (1995). R package version 2.23-6. Software may be obtained from http://CRAN.R-project.org/package=KernSmooth) packages.

### Sample Collection

After an overnight fast, blood was collected in golden cap (Fisher Scientific) serum separator tubes (SST) for lipid analysis and a lavender cap tube with an EDTA additive (Fisher Scientific) for HbA_{1c} and CBC analysis as previously described (Curtis et al., 2010, Cytometry B Clin Cytom 78(5):329-37) using a 21-gauge needle. Four sodium citrate vacutainer tubes were filled with 3 ml of peripheral blood for MP analysis and 30 ml of peripheral blood were also drawn into a 60 ml heparin-coated syringe for the EPC analysis.

### EPC Flow Cytometry

Within one hour after sample collection, 30 ml of whole blood was lysed with ammonium chloride, washed twice with 3% FCS in PBS and resuspended in 10 ml of 3% FCS in PBS. 8×10⁶ cells were incubated with Mouse IgG (Sigma, Cat#15381-10MG) for 10 minutes on ice. After blocking, cells were stained with: 20 μl FITC-CD31 (BD Cat#555445, Clone WM59), 20 μl PE-Cy7-CD34 (BD Cat#348791, Clone 8G12), 20 μl Percp-Cy5.5-CD3 (BD Cat#340949, Clone SK7), 20 μl Percp-Cy5.5-CD33 (BD Cat#341650, Clone P67.6), 20 μl Percp-Cy5.5-CD19 (BD Cat#340951, Clone SJ25C1), 5 μl V450-CD45 (BD Cat#560367 Clone HI30), 10 μl PE-CD133 (Miltenyl Biotec Cat#130-080-801, Clone AC133), and 24 μl APC-VEGFR2 (R&D Cat# FAB357A, Clone 89106) for 45 minutes on ice in the dark. Fluorescence minus one tubes were used for setting up the negative gates (Roederer, 2001, Cytometry 45:194-205). After staining, samples were washed twice and 600 μl of PBS with 0.1% of BSA and 5 μg/ml of Propidium Iodide (Sigma cat# P4170) was added to each tube.

Compensation tubes were prepared with BD CompBeads (anti-mouse IgG and negative control, Cat#552843). 8 peak fluorescent calibration beads (Spherotech, cat# RCP-30-SA) were run before and after acquisition each day for normalization between days. All acquisition occurred on a BD FACS Canto A analytical flow cytometer and stopped after at least 200,000 lymphocytes were counted.

### EPC Data Analysi

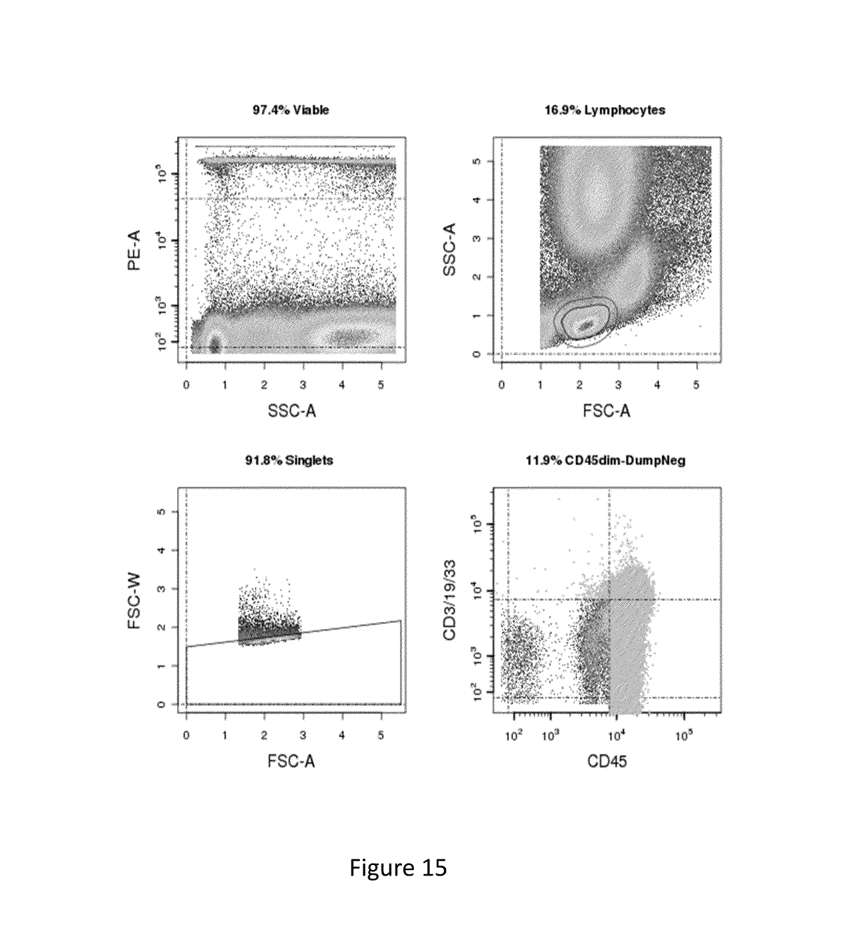

For each subject, the list mode data were read and processed using the flowCore Bioconductor package (Ellis, B., Haaland, P., Hahne, F., Le Meur, N., and Gopalakrishnan, N. 2009. flowCore: Basic Structures for flow cytometry data. Bioconductor package version 1.18.0. Software may be obtained from http://bioconductor.org/packages/2.10/bioc/html/flowCore.html) in untransformed linear coordinates. Digital compensation was applied based on the spillover matrix determined by the FACSDiva acquisition software and stored in the FCS header, and data were normalized based on reference beads (Spherotech, Cat# RCP-30-5A) run each day using the brightest peak. The fluorescence data were biexponentially transformed and the scattering data were linearly transformed to put the fluorescence and scattering data on a similar scale. A fully automated gating strategy was developed in order to eliminate possible operator bias (FIG. 15). Briefly, events whose Forward Scatter Area (FSC-A) or Side Scatter Area (SSC-A) signals above the region of interest and small events on Forward Scatter Area (below the lymphocyte cluster) were removed to prevent interference with automated gating. A viability gate was applied in which all events above a constant threshold on the PE-A (Phycoerythrin) detector (on a 575/26 band pass filter), representing PI binding, were excluded. An automated polygon gate using a blob analysis algorithm based on a 2D kernel density estimate was used to detect events in the lymphocyte region in FSC-A versus SSC-A. An automated gate was created to select singlet cells based on the fact that the doublet population is separated from the singlet population on FSC-A versus Forward Scatter Width (FSC-W). Finally a two-dimensional rectangular gate in CD45 versus lineage cocktail (CD3 T-Cell, CD19 B-Cell, and CD33 Monocyte) was created to eliminate cells that have already differentiated into the hematopoetic lineage and/or cells that express CD45 brightly, leaving only lineage^{negative} and CD45^{dim-negative} events. The events that remained were then analyzed using the flowFP package (Holyst, H. A., and Rogers, W. T. 2009. FlowFP: Fingerprinting for Flow Cytometry. Bioconductor Package version 1.12.1. Software may be obtained from http://bioconductor.org/packages/2.10/bioc/html/flowFP.html). A binning model was constructed using the method flowFPModel with default resolution based on the aggregate of all gated events from healthy control subjects using the four measured fluorescence parameters not used in the gating (PE-Cy7-CD34, PE-CD133, APC-VEGF-R2 and FITC-CD31). The resulting binning model contained 1024 bins. Fingerprints were then generated using the method flowFP for all of the samples from both DM and HC subjects. Relative event counts in each bin were computed by dividing the number of events in the bin by the number of events in the small cell gate (generally regarded as representing the number of lymphocytes measured in the flow cytometer). Absolute event counts were obtained by multiplying the relative event counts by the ALC laboratory result expressed as 1000's of lymphocytes per μl of whole blood. Finally, bins were compared between DM and HC samples using the Wilcoxon test, and P-values were corrected for multiple comparisons using the Benjamimi-Hochberg correction. P-values <0.05 were considered significant.

### MP Isolation

Platelet-poor plasma (PPP) was obtained using centrifugation from blood collected in a sodium citrate tube. Within an hour after blood collection, in order to isolate MPs as previously described (Curtis et al., 2010, Cytometry B Clin Cytom 78(5):329-37), whole blood was centrifuged at 2,500 g for 15 minutes at room temperature. The PPP was carefully moved to a new tube and mixed gently. Fresh samples were then analyzed via flow cytometry.

### MP Flow Cytometry

50 μl PPP was labeled with 2.5 μl FITC-Annexin-V (BD Bioscience Cat#556570), 2.5 μl PE-CD144 (BD Bioscience Cat#560410, clone 55-7H1), 0.75 μl Percp-Cy5.5-CD64 (BD Bioscience Cat#561194, Clone 10.1), 0.75 μl AF647-CD105 (BD Bioscience Cat#561439, clone 266), 0.75 μl APC-H7-CD41a (BD Bioscience Cat#561422, clone HIP8), 2.5 μl PE-Cy7-CD31 (Biolegend Cat#303118, clone WM59) and 0.75 μl BV421-CD3 (Biolegend Cat#300433, Clone UCHT1) for 30 minutes at room temperature in the dark. The antibodies were double-filtered before labeling with a 0.1 μm low protein binding filter (Millipore, Cat# SLVV033RS).

After the sample tubes were stained, 5 μl of 3.0 μm beads were added to each tube as reference counting beads. Annexin Buffer (10 mM Hepes, pH 7.4, 140 mil NaCl, and 2.5 mM CaCl₂) was added to each tube to make the total volume 500 μl. The Annexin Buffer was double-filtered by 0.22 micro filter followed by 0.1 μm filter.

The BD FACSCanto A cytometer was calibrated daily with Cell Tracker Beads (BD) using Diva Software version 6.1.2. Forward and side scatter threshold, photomultiplier tube (PMT) voltage and window extension (WE) were optimized to detect 0.1-1.0 μm particles using 0.3, 1.0 and 3.0 μm calibration beads. Beads of known size (0.3 μm, 1.0 μm and 3.0 μm) were used for the estimation of MP size.

The acquisition was stopped when a fixed number of 3.0 μm beads (20,000) were counted resulting in 82 thousand to 2.2 million MPs per sample. Compensation tubes were also run using PPP, BD CompBead (BD Bioscience Cat#552843), and were stained using the same reagents as were used in the sample tubes.

### MP Data Analysis

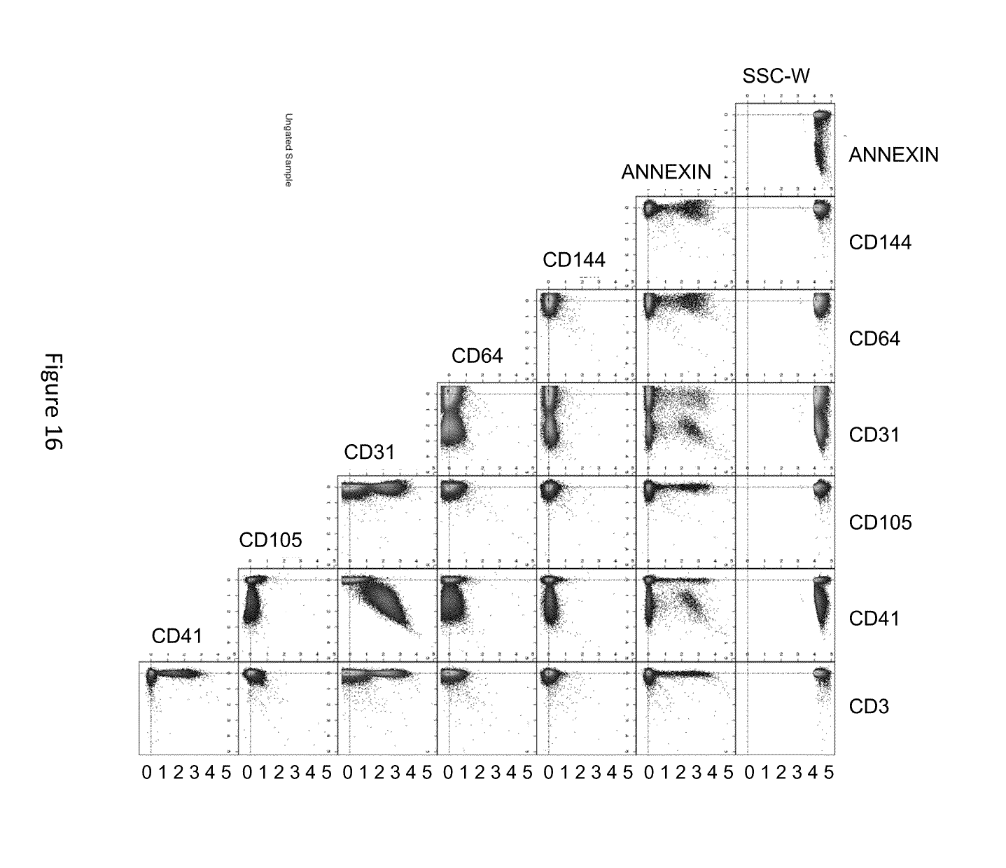

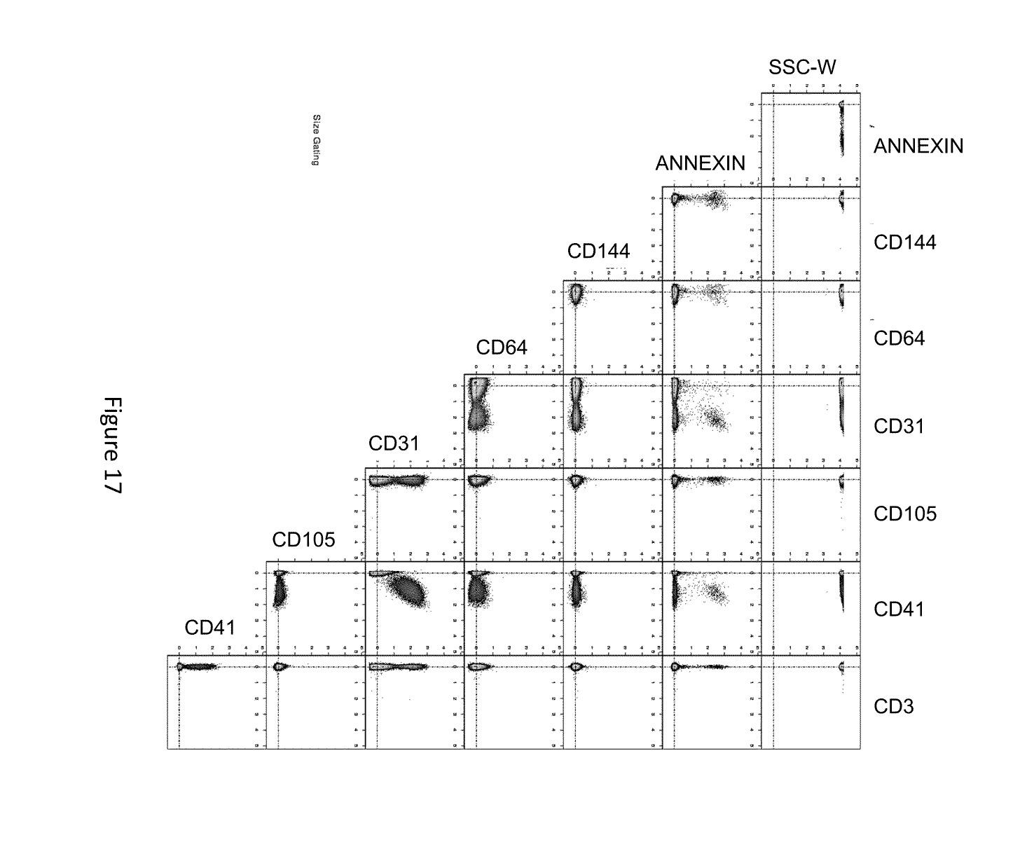

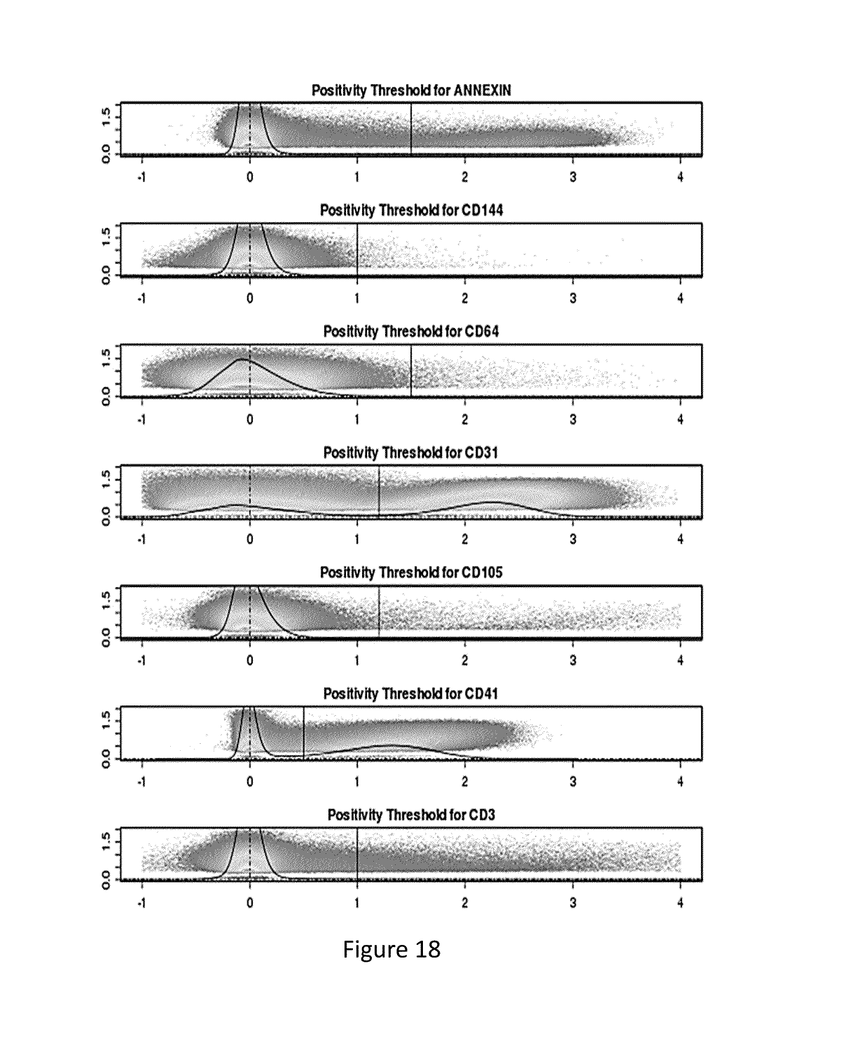

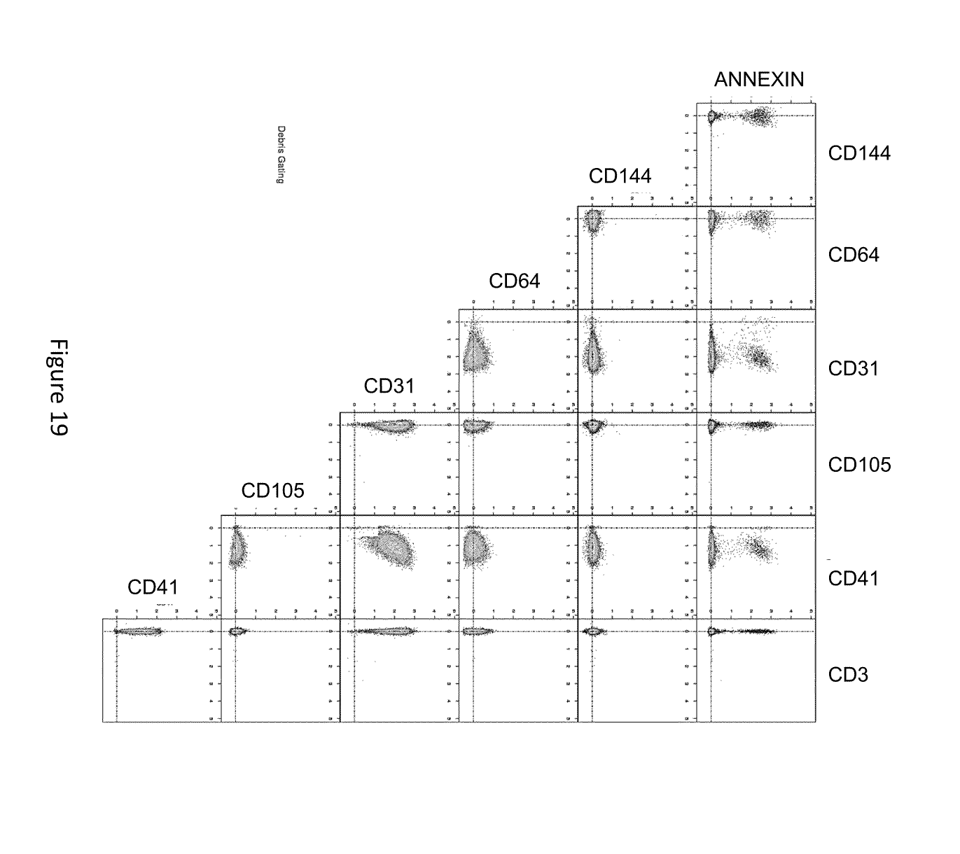

The R environment for statistical computing was used for analysis of the flow cytometry data. The flowCore package (38) was used for reading files, compensation and gating. FlowFP (Rogers et al., 2008, Cytometry Part A 73A:430-441) was used for Cytometric Fingerprinting (CF) analysis. For each subject, the list mode data were read in untransformed linear coordinates. Digital compensation was applied based on the spillover matrix determined by the Diva acquisition software and stored in the FCS header. Data were gated on Side Scatter Width (FIGS. 16 and 17) as a relative measure of particle size to eliminate all events larger than 1 μm as determined by the size calibration beads collected each day. Thresholds for positive expression of markers were identified by examining the kernel density estimates of the univariate distributions of all events captured (FIG. 18). In order to determine the sensitivity of the analysis results to the choice of thresholds, the thresholds for fluorescence markers without clear separation in the kernel density estimate between the positive and negative populations were increased by various factors between about 1.4- to 2.5-fold and the results were shown to be stable, demonstrating that the choice of thresholds did not materially affect the results. The fluorescence data were biexponentially transformed and events that expressed none of the markers in the panel were presumed to be debris and were gated out (FIG. 19). Fingerprinting analysis was carried out on resulting distributions using the R package flowFP (Holyst, H. A., and Rogers, W. T. 2009. FlowFP: Fingerprinting for Flow Cytometry. Bioconductor Package version 1.12.1. Software may be obtained from http://bioconductor.org/packages/2.10/bioc/html/flowFP.html). A binning model was created using the method flowFPModel based on the HC subjects using all fluorescence markers at default resolution, resulting in 8192 bins. Fingerprints were then generated for each sample using the methodflowFP based on this model. Bins were compared between DM and HC samples using the Wilcoxon test, corrected for multiple comparisons using the Benjamimi-Hochberg correction. Corrected P-values of <0.05 were considered significant. Bins judged to be significant were further grouped by phenotypic similarity. Thus, a phenotypic subset determined to be differentially expressed between DM as compared with HC can be comprised of one or more bins adjacent in multivariate space, all of which may fall on the same side of each parameter's threshold for positive expression.

### High Sensitivity C-Reactive Protein Measurement

High-sensitivity C-reactive protein was measured using a laser-based immunonephelometric quantitation method on an automated laser-based nephelometer (Siemens Healthcare Diagnostics, Model BNII) as per manufacturers suggested methods.

### Statistical Analysis

Participant characteristics were compared between DM and HC groups using Wilcoxon rank-sum tests or Fisher's exact tests, as appropriate. EPC and MP counts were compared between DM and HC groups using Wilcoxon rank-sum tests. Multivariable linear regression models were used to estimate adjusted differences in EPC and MP counts between groups. Adjustment variables were selected based on a stepwise model-selection procedure based on the Akaike information criterion (AIC), for which a variable that reduced the AIC was retained. Variables evaluated were: age, gender, race, current exercise, and body mass index. Because EPC and MP counts were positively skewed, a log transformation was applied such that the exponentiated regression coefficient for DM versus HC quantified the ratio in the average count between DM and HC groups. In a post-hoc analysis, the MP and EPC counts that exhibited the strongest differences between DM and HC groups were each standardized by the median in the HC group. A plot of the standardized EPCs versus the standardized MPs was used to graphically evaluate whether a combination of MPs and EPCs was useful in distinguishing DM from HC. More formally, receiver operating characteristic (ROC) curves were used to evaluate the ability of the MP and EPC counts, with and without hsCRP, to discriminate between DM and HC groups. All analyses were completed using R.

### Example 6

### Results

The DM group was somewhat older than the HC group, as shown in Table 5.

TABLE 5
Participant characteristics

| | EPC cohort | | | | | Microparticle cohort | | | | |
|---|---|---|---|---|---|---|---|---|---|---|
| | Diabetics | | Controls | | | Diabetics | | Controls | | |
| | n = 62 | | n = 51 | | P* | n = 48 | | n = 48 | | P* |
| Demographic Characteristics | | | | | | | | | | |
| Age, years | 67 | (58, 73) | 59 | (55, 68) | 0.019 | 66 | (59, 72) | 59 | (55, 67) | 0.010 |
| Female, n (%) | 26 | (42) | 32 | (63) | 0.037 | 19 | (40) | 30 | (63) | 0.041 |
| Black or African- | 33 | (53) | 12 | (24) | 0.002 | 26 | (54) | 12 | (25) | 0.006 |
| American, n (%) | | | | | | | | | | |
| Smoking status, n | | | | | <0.001 | | | | | <0.001 |
| (%) | | | | | | | | | | |
| Current | 14 | (23) | 0 | (0) | | 11 | (23) | 0 | (0) | |
| Former | 38 | (61) | 18 | (35) | | 30 | (63) | 16 | (33) | |
| Never | 10 | (16) | 33 | (65) | | 7 | (15) | 32 | (67) | |
| Regular exercise, n | 34 | (55) | 42 | (82) | <0.001 | 26 | (54) | 38 | (79) | <0.001 |
| (%) | | | | | | | | | | |
| Body mass index, | 30 | (27, 36) | 24 | (23, 26) | <0.001 | 31 | (27, 36) | 24 | (23, 26) | <0.001 |
| kg/m² | | | | | | | | | | |
| Tonsillectomy, n | 21 | (34) | 25 | (49) | 0.13 | 17 | (35) | 24 | (50) | 0.22 |
| (%) | | | | | | | | | | |
| Laboratory Values | | | | | | | | | | |
| Blood pressure, | | | | | | | | | | |
| mmHg | | | | | | | | | | |
| Systolic | 136 | (120, 150) | 122 | (111, 129) | <0.001 | 133 | (121, 148) | 122 | (111, 131) | <0.001 |
| Diastolic | 78 | (70, 85) | 77 | (72, 85) | 0.93 | 78 | (70, 85) | 77 | (72, 86) | 0.80 |
| Cholesterol level, | | | | | | | | | | |
| mg/dL | | | | | | | | | | |
| Total | 154 | (126, 201) | 211 | (186, 226) | <0.001 | 145 | (126, 192) | 211 | (185, 225) | <0.001 |
| Low-density | 80 | (66, 106) | 127 | (117, 148) | <0.001 | 75 | (65, 100) | 127 | (116, 147) | <0001 |
| lipoprotein | | | | | | | | | | |
| High-density | 38 | (32, 48) | 58 | (49, 73) | <0.001 | 37 | (32, 49) | 59 | (49, 74) | <0.001 |
| lipoprotein | | | | | | | | | | |
| High-sensitivity C- | 2.4 | (1.0, 6.8) | 0.9 | (0.4, 1.5) | <0.001 | 2.4 | (1.0, 7.0) | 0.9 | (0.4, 1.6) | <0.001 |
| reactive protein, | | | | | | | | | | |
| mg/L | | | | | | | | | | |
| Hemoglobin A1c, | 7.0 | (6.5, 8.8) | 5.6 | (5.5, 5.7) | <0.001 | 7.0 | (6.5, 8.7) | 5.6 | (5.5, 5.7) | <0.001 |
| % | | | | | | | | | | |
| Absolute | 1.8 | (1.2, 2.4) | 1.4 | (1.2, 1.8) | 0.009 | 1.8 | (1.1, 2.2) | 1.3 | (1.2, 1.7) | 0.045 |
| lymphocyte count, | | | | | | | | | | |
| 10³ | | | | | | | | | | |
| cells/microliter | | | | | | | | | | |
| Medication Use | | | | | | | | | | |
| Antiplatelet, n (%) | 45 | (73) | 5 | (10) | <0.001 | 37 | (77) | 3 | (6) | <0.001 |
| Statin, n (%) | 42 | (68) | 0 | (0) | <0.001 | 35 | (73) | 0 | (0) | <0.001 |

summaries presented as median (inter-quartile range) unless otherwise noted as n (%)
*P values obtained from Wilcoxon rank-sum tests or Fisher's exact tests, as appropriate

Gender differed slightly between the DM and HC with ˜60% females in HC group compared to ˜40% females in DM group. There were also higher numbers of African Americans in the DM group compared to the HC group. In addition, there was a higher proportion of smoking, lower proportion reporting exercise, and higher average BMI in the DM group. As expected, HbA_{1c} was elevated in the DM group, as was blood pressure; however, LDL levels were lower in DM compared to controls as 68% of the DM cohort for the EPC analysis and 73% of the DM cohort in the MP analysis were on statins as compared with none of the controls. Interestingly, the LDL of the DM patients who were not on statins was higher than those on statins, but still lower than the HC. This is not entirely unexpected as it is theorized that while LDL for DM patients may be similar to HC, the composition of the LDL is different and more atherogenic (Nesto, 2008, Clinical Diabetes 26:8-13). Additionally, levels of HDL were lower in the DM group compared to the HC group. Levels of high-sensitivity CRP were higher in DM than HC in both the MP and EPC cohorts (Haffner, 2006, Am J Cardiol 97(2A):3A-11A). While most of the DM patients were on antiplatelet and/or statin drugs, few of the controls were on preventative antiplatelet medications.

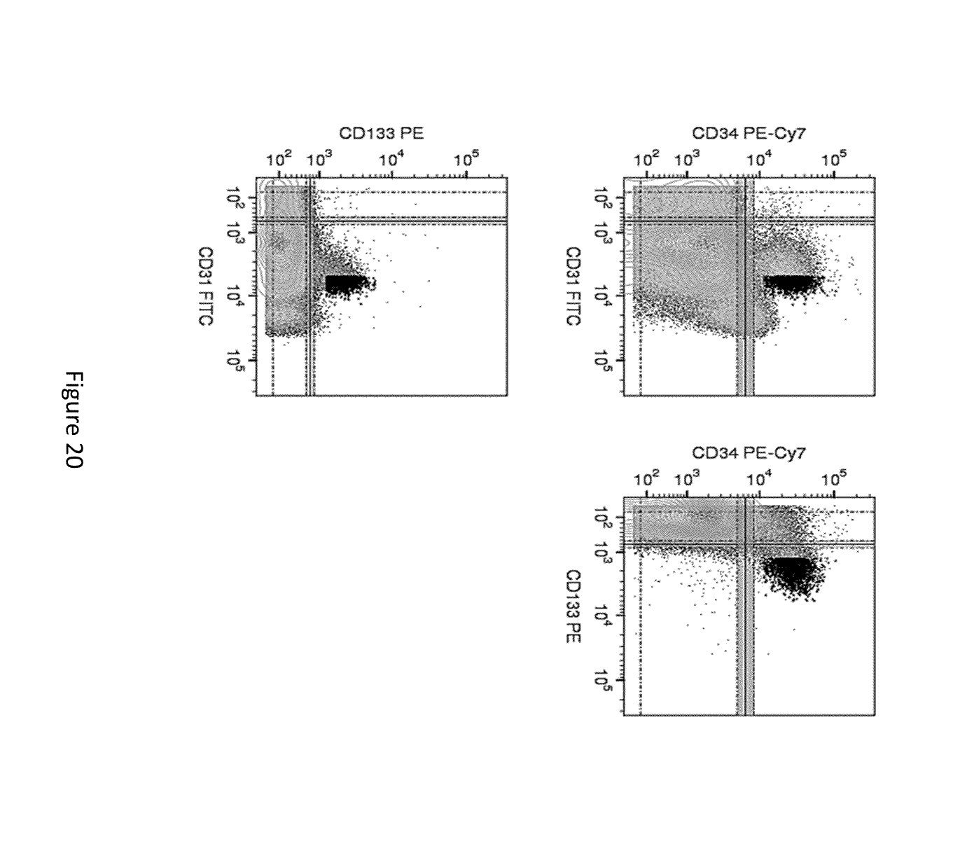

Assessment of EPCs via a traditional manual sequential gating analysis (using FlowJo, Treestar, Ashland, Oreg.) demonstrated no statistically significant differences between DM and HC. However, when cytometric fingerprinting was applied, a distinct phenotypic subset, referred to herein as EPCs, was discerned in one fingerprint bin. This subset had the phenotype CD34⁺/CD31⁺/CD133^{bright}/CD45^{dim-negative} (FIG. 20), and was lower on average in DM patients compared with HC. The relative event count (EPC^{rel}) of this subset was significantly different between DM and HC, even after adjustment for covariates as discussed above. Separately, the ALC level was significantly different between diabetics and controls (P<0.001), but this difference was attenuated after adjustment for confounding factors (P=0.11). This finding is consistent with the work of other authors who found that lymphocyte counts were associated with cardiovascular events; however, there was no correlation after adjustment for confounding variables (Eryd et al., 2012, Arterioscler Thromb Vasc Biol. 32(2):533-9).

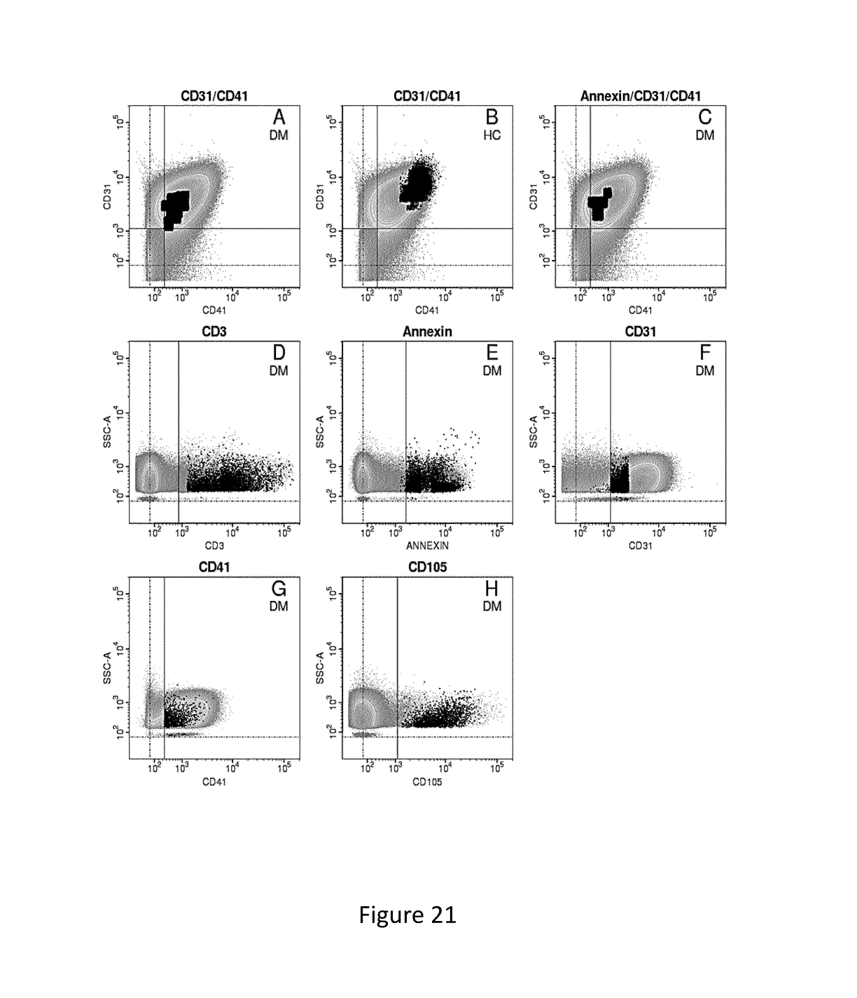

Assessment of MPs via cytometric fingerprinting led to the discovery of 8 different phenotypic subsets of MPs that differed significantly between HC and DM groups (FIG. 21). In all of these except one (discussed below), concentrations were higher on average in DM as compared with HC. Each statistically significant population discovered via fingerprinting is actually a subset of the MPs positive for the indicated marker(s). For example, FIG. 21G shows a subset of MPs that are positive for CD41. These are not all of the MPs that are positive for CD41, but rather those events that are positive for CD41 while also being negative for all other markers in the panel and that fall into fingerprint bins that were significantly differently populated between the DM and HC cohorts. One subset that differed strongly between DM and HC was the subset of CD3⁺ T-lymphocyte MPs (TMP), which was also present at higher concentrations in DM patients compared with HC. Similarly, a subset of the CD 105⁺ Endothelial MP (EMP) population was at higher average concentration in DM patients. Another significant subset is comprised of events that are Annexin V⁺, which was up-regulated in DM. While the Annexin V single positive subset signifies an apoptotic MP, it is not a marker that is specific for the parent cell type. Another subset that was increased was the CD31⁺ phenotype. While this subset had a p-value >0.05, it was significant after adjustment for confounding variables. PE-CAM1 (CD31) marker alone is not specific for one type of cell. The last 4 subsets of MP that were found to be significant were all platelet MP (PMP) and were positive for CD41 (and some for CD31 as well). The CD41 single positive population and Annexin V⁺/CD31⁺/CD41⁺ triple positive subsets were both marginally significant (P<0.05) and up-regulated in DM patients. Neither the CD41⁺ nor the Annexin V⁺/CD31⁺/CD41⁺ subsets were significant after adjustment for confounding variables. Finally, there were two CD31⁺/CD41⁺ double-positive subsets of PMP that were discovered by fingerprinting to differ between DM and HC. One of these was marginally significant (P=0.11) and upregulated in DM patients (CD31^{dim}/CD41^{dim}) while the other was highly significant (P<0.001) and was the only one of the differentially expressed subsets that was present at lower concentrations on average in DM compared with HC (CD31^{bright}/CD41^{bright}). Finally, the ratio of the CD31^{dim}/CD41^{dim} subset to the CD31^{bright}/CD41^{bright} subset was more strongly differentially expressed (P<0.001) than either the dim or the bright subsets individually, or any of the other differentially expressed MP subsets discovered by cytometric fingerprinting, and was consequently used in forming the combined measure of EPCs and MPs of the present invention.

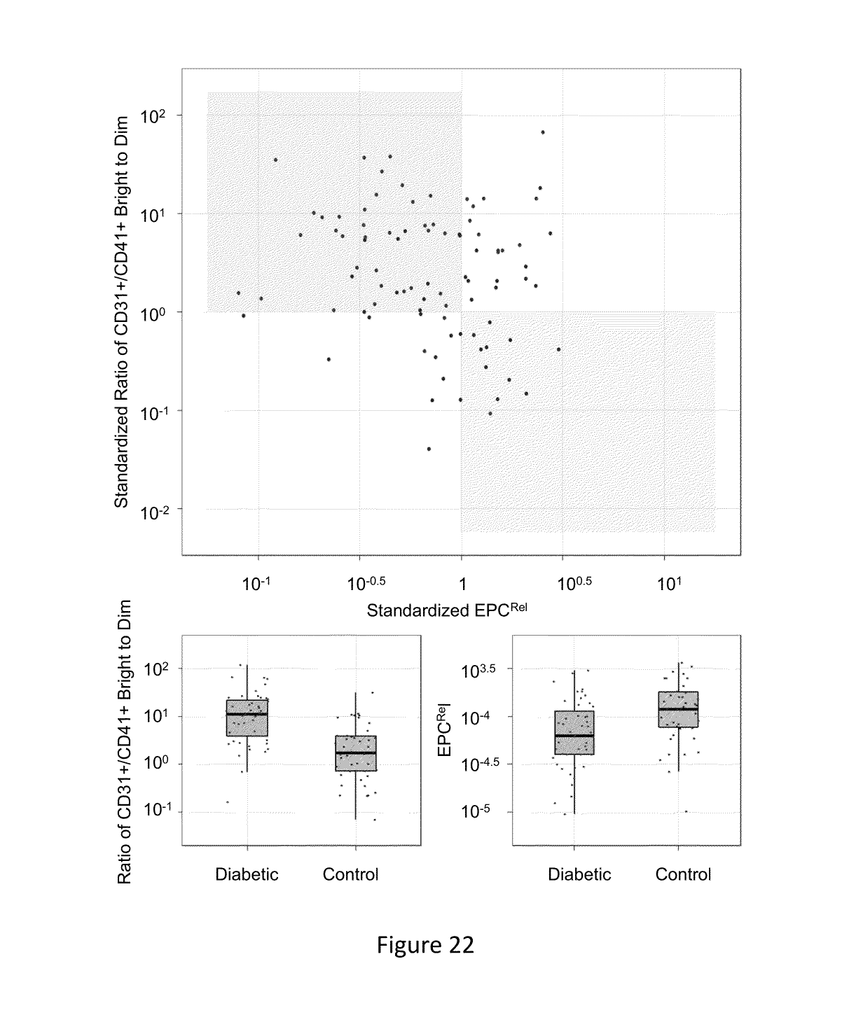

As it is generally thought that progenitor cells play a role in cellular reparative capacity and new vascular growth, and microparticles are a measure of cellular damage, it was hypothesized that a combination of the two would be more clinically informative with respect to cardiovascular status than either one alone. The ability of relative event counts of EPCs (CD31⁺, CD34⁺, CD133^{bright}, CD45^{dim-negative}) per lymphocyte and by the ratio of dim to bright CD31⁺/CD41⁺ microparticles was evaluated to discriminate between DM and HC (FIG. 22). The area under the ROC curve (AUC) for a combination of these two markers was 0.86, 95% CI: (0.79, 0.94), indicating high discrimination accuracy. When combined with CRP, the AUC increased to 0.90, 95% CI: (0.83, 0.96). Table 6 illustrates the comparison of EPC and MP subsets between DM and HC groups.

TABLE 6
Comparison of EPC and MP subsets between DM and HC groups

| | | | |
|---|---|---|---|
| Diabetics | Controls | | Adjusted |
| Median | Median | | ratio** |
| (IQR) | (IQR) | P* | (95% CI) |

Endothelial Progenitor Cells

| | | | | | | | |
|---|---|---|---|---|---|---|---|
| EPC^{Abs} | 97 | (61, 170) | 165 | (100, 250) | 0.005 | 0.85 | (0.63, 1.2) |
| EPC^{Rel} | 6.1 | (4.0, 9.9) | 12 | (7.5, 18) | <0.001 | 0.65 | (0.49, 0.88) |

Microparticle Counts (per ml plasma)

| | | | | | | | |
|---|---|---|---|---|---|---|---|
| CD3⁺/CD41⁺ Bright | 15 | (8.1, 37) | 51 | (20, 150) | <0.001 | 0.28 | (0.16, 0.49) |
| CD31⁺/CD41⁺ Dim | 120 | (55, 340) | 89 | (50, 180) | 0.11 | 1.4 | (0.86, 2.2) |
| Ratio of CD31⁺/CD41⁺ | 11 | (3.1, 21) | 2.1 | (0.72, 4.2) | <0.001 | 6.5 | (3.5, 12) |
| Dim to Bright | | | | | | | |
| Annexin⁺ | 4.2 | (2.9, 7.1) | 2.3 | (1.6, 3.2) | <0.001 | 1.6 | (1.2, 2.1) |
| CD31⁺ | 10 | (5.6, 27) | 7.8 | (3.3, 14) | 0.058 | 1.7 | (1.1, 2.8) |
| CD41⁺ | 0.38 | (0.20, 0.99) | 0.20 | (0.051, 0.66) | 0.027 | 1.6 | (1.0, 2.5) |
| Annexin⁺/CD31⁺/CD41⁺ | 4.9 | (1.9, 13) | 3.2 | (1.6, 6.6) | 0.031 | 1.4 | (0.88, 2.4) |
| CD3⁺ | 3.1 | (1.5, 4.4) | 0.98 | (0.60, 2.4) | <0.001 | 2.5 | (1.5, 4.3) |
| CD105⁺ | 1.9 | (1.3, 3.8) | 1.2 | (0.69, 2.1) | 0.001 | 1.9 | (1.3, 2.6) |

IQR, inter-quartile range
*P values obtained from Wilcoxon rank-sum tests
**Obtained from multivariable linear regression models for log-transformed counts

The study results identified one sub-population of CD31⁺/CD34⁺/CD45^{dim-negative}/CD133^{bright} EPCs up-regulated in HC compared to DM. In addition, it was determined that there were 7 subpopulations of microparticles corresponding to platelet, T-lymphocyte, Annexin V⁺, and endothelial microparticles. Therefore, this study confirms the hypothesis that levels of EPCs and MPs are different between HC and DM patients with atherosclerosis, and suggests that these measures are useful as prognostic markers for cardiovascular health.

The use of EPCs as a biomarker is significant as it is directly involved in pathological processes in the cardiovascular system as opposed to other commonly used biomarkers, which respond non-specifically to the underlying condition. In the case of DM patients vs HC, there was one population of EPCs, with a phenotype of CD31⁺/CD34⁺/CD45^{dim-negative}/CD133⁺ that was upregulated in the control group. This EPC population is similar to the circulating hematopoietic stem and progenitor cells (CHSPC) population described by Estes et. Al. (Estes et al., 2010, Cytometry Part A 77A:831-839) and shown in FIG. 20. EPCs are a heterogeneous population whose specific phenotypic definition remains controversial. Generally, EPC phenotypes described in the literature will include a stem cell marker such as CD34, an immaturity marker such as CD133, and an endothelial marker such as VEGF-R2 (KDR) (Möbius-Winkler et al., 2009, Cytometry Part A 75A:25-37).

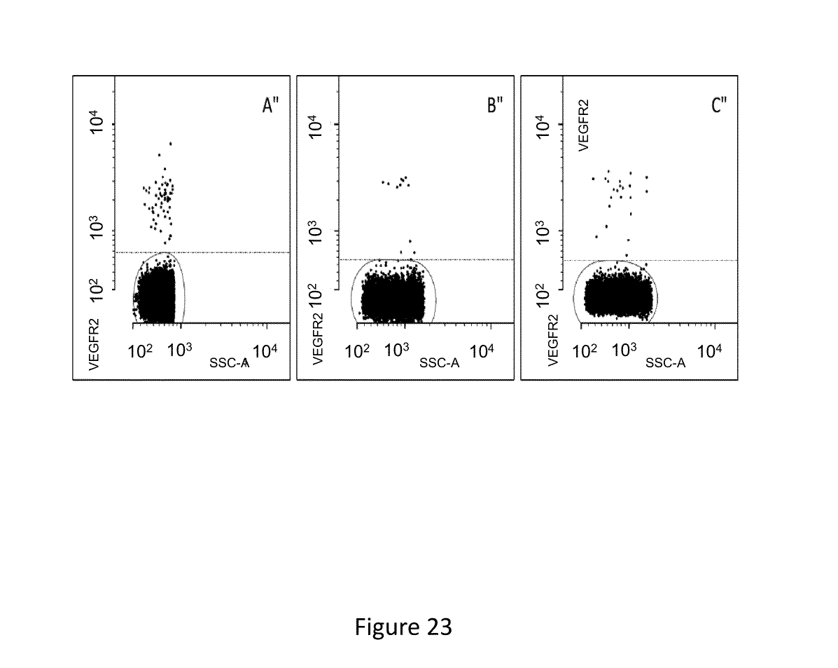

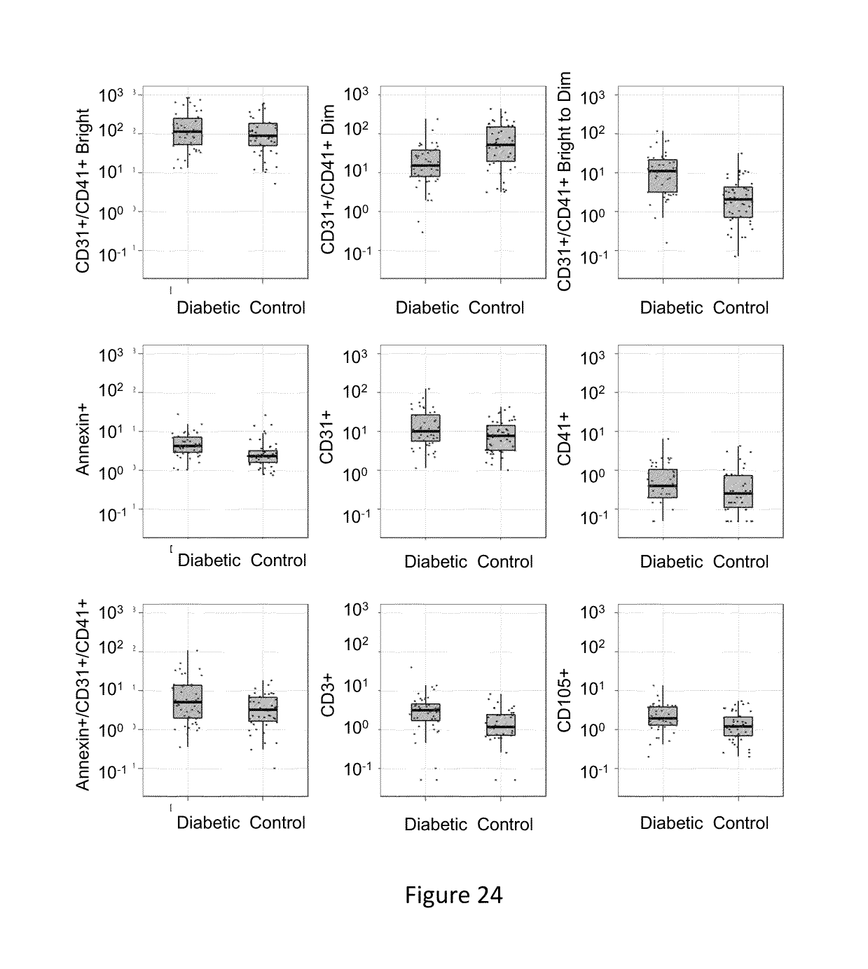

In this study, a comprehensive panel was employed, in which cells were first selected based on size. Then, cells belonging to the mature hematopoietic lineage were removed by gating out CD3⁺, CD19⁺, CD33⁺ and CD45^{bright} cells. Then, using fingerprinting, the remaining population was subdivided and subjected to statistical evaluation, without any predetermined bias, to discover if there were populations of cells differentially expressed between the DM patients and HC. As with some other studies, VEGF-R2 was found to not be a useful marker in analysis (Estes, M. L., Mund, J. A., Ingram, D. A., and Case, J. 2001. Identification of Endothelial Cells and Progenitor Cell Subsets in Human Peripheral Blood. In Current Protocols in Cytometry: John Wiley & Sons, Inc.), indicating that either the marker is not informative or, as some studies have shown, the reagent is not reliable. As can be seen in FIG. 23, there are many false positive events for VEGF-R2, and therefore it is hard to find a true threshold for positivity. Additionally, it has been shown that VEGF-R2 antibody cannot be titrated sufficiently as an increase in concentration of antibody in the panel increases the resulting signal. The panel in the present study contained an additional endothelial marker (CD31), which was also positively expressed in the informative phenotype, supporting a similar general phenotype of immature (CD133), stem (CD34), and endothelial (CD31) markers that are accepted to be expressed on EPCs.

The population found to be differentially expressed in the present study expresses the same markers found in an Estes et. al. study (Estes et al., 2010, Cytometry Part A 77A:831-839) that were shown to be pro-angiogenic. In Estes et. al., an in vivo tumor model in mice that showed cells with the same phenotype as the EPCs in this study resulted in a significant increase in tumor growth, indicating that these cells are involved in neo-angiogenesis. Additionally, a similar population of cells CD34⁺/CD133⁺/CD117⁺ (CD117, c-Kit, is a stem cell marker), were found to produce increased angiogenesis in ischemic tissue resulting in 3-5 fold higher capillary numbers in infarct zones in rats after 2 weeks as opposed to more mature vascular endothelia not expressing CD133 (Kocher et al., 2001, Nat Med 7:430-436). The present study did not include CD117, however, it is likely due to the two other markers that the two populations overlap and share similar function. Therefore, the unbiased results of our study are consistent with other studies in showing that pro-angiogenic EPCs are differentially expressed between an atherosclerotic population and HC.

Platelet MPs (PMP) are known to have both beneficial and detrimental effects on vascular health (Tushuizen et al., 2011, Arterioscler Thromb Vasc Biol 31:4-9). These claims are supported by the present study as multiple distinct PMP populations were significantly different between the two populations, three of which were up-regulated in DM patients and the other up-regulated in HC. Omoto et. al. (Omoto et al., 1999, Nephron 81:271-277) found that PMPs are significantly up-regulated in type 2 DM patients with nephropathy compared to DM patients without complications, suggesting that PMPs are involved in activity leading to the kidney dysfunction. Furthermore, Tan et. al. (Tan et al., 2005, Diabetic Medicine 22:1657-1662) discovered that DM patients with clinically apparent atherosclerosis had a significantly higher level of PMPs than both DM patients without clinically apparent atherosclerosis and HC. Additionally, both PMPs and EMPs were shown to be up-regulated in patients with severe hypertension (Preston et al., 2003, Hypertension 41:211-217) suggesting that EMPs and PMPs may be markers for effects of blood pressure on endothelium and thus vascular injury. PMPs have also been shown to stimulate endothelial cells in vitro to release cytokines and express adhesion molecules (Nomura et al., 2001, Atherosclerosis 158:277-287). The present results demonstrate that the majority of MP subsets are markers of poor vascular health. However, the unbiased cytometric fingerprinting method was also able to discover a population of PMPs that were up-regulated in HC, which is in keeping with other findings that PMPs can have beneficial effects. PMP have been shown to aid in the angiogenic activity of human umbilical vein endothelial cells and augmented EPC differentiation in peripheral blood mononuclear cells (Kim et al., 2004, Br J Haematol 124(3):376-384). It has also been shown by Mause et al that PMPs can boost the potential for angiogenic early outgrowth cells to restore endothelial integrity after vascular injury (Mause et al., 2010, Circulation 122:495-506). Therefore, through the unbiased computational approach this study distinguished two separate populations of PMPs that play a role in vascular health.

Endothelial MPs (EMP), like PMPs, are elevated in DM patients and it has been theorized that EMPs are associated with vascular dysfunction and, are a sign of cellular apoptosis, and therefore reflecting vascular wall damage (Chironi et al., 2009, Cell Tissue Res 335:143-151). In one study, EMP levels were negatively correlated with flow-mediated dilation (FMD) indicating that EMPs are associated with endothelial dysfunction (Feng et al., 2010, Atherosclerosis 208:5). Additionally, another study showed, EMPs were significantly higher in patients with coronary artery disease than in controls (Bernal-Mizrachi et al., 2003, American Heart Journal 145:962-970). The CD105⁺ population subset that was discovered in this study likely corresponds to EMPs, which has been shown in numerous studies to be a marker for disease and vascular dysfunction. Therefore the results presented herein are in concordance with previous work on EMPs showing that DM patients with clinical atherosclerosis have higher levels of EMPs than HC.

The results also showed that a CD3⁺ T-cell MP (TMP) population subset was significantly up-regulated in DM patients compared to HC. This finding supports previous work, as TMPs are known to be pro-inflammatory, decreasing NO production by reducing the level of eNOS expression and increasing oxidative stress in endothelial cells (Martin et al., 2004, Circulation 109:1653-1659). Additionally, TMPs induce endothelial dysfunction in both conductance and resistance arteries by alteration of NO prostacyclin pathways. Furthermore, TMPs impaired acetylcholine-induced relaxation of aortic rings in similar concentrations to humans (Martin et al., 2004, Circulation 109:1653-1659). Finally, TMPs also have been shown to produce endothelial dysfunction in response to flow and chemical stimuli (Martin et al., 2004, Circulation 109:1653-1659).

Annexin V⁺ cells bind to phosphatidylserine, which is a marker of apoptosis. Studies done on atherosclerotic plaques have shown that apoptotic MPs found in plaques account for almost all of the TF (tissue factor) activity of the plaque extracts. This indicates that the MPs may play a role in the initiation of the coagulation cascade (Mallat et al., 1999, Circulation 99:348-353). Additionally, MPs positive for Annexin V are significantly upregulated in patients with acute coronary syndrome compared to patients with stable angina (Mallat et al., 2000, Circulation 101:841-843). This signifies that an increase Annexin V⁺ MPs reflects a worsening of the atherosclerotic condition of patients. However, a study limitation was that the prothrombinase assay on Annexin V⁺ MP was done separately from the flow cytometry, therefore these cells could be of any origin. The present study supports these findings as the Annexin V⁺ population subset was significantly up-regulated in DM patients.

Cell-based systems analyses, also known as ‘cytomics’, integrates the biologic consequences of environmental and genetic cardiovascular risk factors. There is an unmet clinical need to develop such assays that could be used routinely to guide medical therapy and risk assessment. The present results provide a comprehensive insight into vascular health by using pattern discovery computational methods to analyze characteristics of several targets, including populations of vascular microparticles (recently identified as robust biomarkers of vascular health) and endothelial progenitor cells. For example, asymptomatic patients can be evaluated for cardiovascular risk, and symptomatic patients can be monitored longitudinally. These capabilities realize the main goals of personalized medicine.

The purpose of this study was to use an unbiased approach to find phenotypic subsets of cells and microparticles that were differentially expressed between an atherosclerotic DM population and HC. As such, no functional assays were conducted on either the EPC (such as ECFC or in vivo animal models) or MP (such as a prothrombinase assay) populations that were discovered. Suggested functions of these populations are derived from research done by other groups that are cited in the discussion.

Still, unlike other studies, the use of cytometric fingerprinting and a broad assay allowed us to remove any unintentional gating biases to find numerous populations that were differentially expressed in the populations. Furthermore, the methods used involved rigorous flow cytometry protocol involving: careful standardization of instruments over time using bead standards with additional residual instrument variation mathematically corrected using the bead standards, use of FMO controls for positivity, biexponential transforming, and use of digital instrumentation. Finally, DM patients and HC were consecutively recruited as opposed to retrospectively.

The results of this study indicate that EPCs are higher, and most MP subsets are lower in healthy controls compared to a DM population with atherosclerosis. Importantly, these results were obtained with cytometric fingerprinting, a novel unbiased method of data analysis capable of discovering distributional patterns that may remain hidden when using conventional methods of analysis. Several subsets were significantly differentially expressed in the two populations, some of which are supported in the literature and others are novel findings. Interestingly, VEGF-R2, a marker that is commonly associated with EPCs, was uninformative. Cytometric fingerprinting is an objective, comprehensive and labor saving method, and has general utility in the analysis of complex, multivariate distributions that may contain hidden data patterns. Taken together these results provide the basis for a vascular health profile, which may be useful for a number of applications including drug development, clinical risk assessment and companion diagnostics.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of determining vascular health in a subject, comprising:
obtaining a biological sample from the subject;
obtaining microparticle data based on the level of at least one set of microparticles in the biological sample;
obtaining progenitor cell data based on the level of at least one set of progenitor cells in the biological sample;
generating a cytometric fingerprint of the biological sample based on the microparticle and progenitor cell data; and
determining the vascular health of the subject based on the generated cytometric fingerprint.

2. The method of claim 1, wherein the level of the at least one set of microparticles is measured by flow cytometry.

3. The method of claim 1, wherein the biological sample is a plasma sample.

4. The method of claim 1, wherein the subject is a subject with psoriasis.

5. The method of claim 1, wherein the subject is a subject with lupus.

6. The method of claim 1, wherein the subject is a subject with known CVD risk factors.

7. The method of claim 1, wherein the subject is a subject with no known CVD risk factors.

8. The method of claim 1, wherein the subject is a diabetic subject.

9. The method of claim 1, wherein the subject is a diabetic subject.

10. The method of claim 9, wherein the subject is a Type 1 diabetic subject.

11. The method of claim 9, wherein the subject is a Type 2 diabetic subject.

12. The method of claim 1, wherein the progenitor cells are endothelial progenitor cells (EPCs).

13. The method of claim 1, wherein when the level of at least one of the microparticle sets is up-regulated, the subject is at risk of cardiovascular disease or vascular dysfunction or of progressing cardiovascular disease or vascular dysfunction.

14. The method of claim 1, wherein when the level of at least one of the progenitor cell sets is down-regulated, the subject is at risk of cardiovascular disease or vascular dysfunction or of progressing cardiovascular disease or vascular dysfunction.

15. The method of claim 1, wherein when the level of at least one of the microparticle sets is up-regulated and the level of at least one of the progenitor cell sets is down-regulated, the subject is at risk of cardiovascular disease or vascular dysfunction or of progressing cardiovascular disease or vascular dysfunction.

16. A method for determining a risk associated with cardiovascular disease or vascular dysfunction in a subject, comprising:
obtaining a biological sample from the subject;
obtaining microparticle data based on the level of at least one set of microparticles in the biological sample;
obtaining progenitor cell data based on the level of at least one set of progenitor cells in the biological sample;
generating a cytometric fingerprint of the biological sample based on the microparticle and progenitor cell data; and
determining the risk associated with cardiovascular disease or vascular dysfunction of the subject based on the generated cytometric fingerprint.

17. The method of claim 16, wherein the level of the at least one set of microparticles is measured by flow cytometry.

18. The method of claim 16, wherein the biological sample is a plasma sample.

19. The method of claim 16, wherein the subject is a diabetic subject.

20. The method of claim 19, wherein the subject is a Type 1 diabetic subject.

21. The method of claim 19, wherein the subject is a Type 2 diabetic subject.

22. The method of claim 16, wherein the progenitor cells are endothelial progenitor cells (EPCs).

23. The method of claim 16, wherein the generated cytometric fingerprint indicates a cellular damage.

24. The method of claim 16, wherein the generated cytometric fingerprint indicates the integrity of endothelium, a loss of endothelial repair capacity, or a combination thereof.

25. The method of claim 16, further comprising generating a cytometric fingerprint of a healthy control sample, and comparing the generated cytometric fingerprint of the subject's biological sample to the cytometric fingerprint of the healthy control sample.
